(19) United States
(12) Patent Application Publication
(10) Pub. No.: US 2024/0317852 A1
(43) Pub. Date: Sep. 26, 2024

(54) NOVEL DARPIN BASED CD33 ENGAGERS

(71) Applicant: MOLECULAR PARTNERS AG, Schlieren (CH)

(72) Inventors: Nina RESCHKE, Schlieren (CH); Christian REICHEN, Schlieren (CH); Bernd SCHLERETH, Schlieren (CH)

(21) Appl. No.: 18/281,156

(22) PCT Filed: Mar. 9, 2022
(86) PCT No.: PCT/IB2022/052120

§ 371 Date: Sep. 8, 2023

Related U.S. Application Data
(60) Provisional application No. 63/158,539, filed on Mar. 9, 2021.
(60) Provisional application No. 63/172,818, filed on Apr. 9, 2021.
(60) Provisional application No. 63/265,179, filed on Dec. 9, 2021.

(51) Int. Cl. C07K 16/28; A61K 38/00; A61K 39/00; A61P 35/00

(52) U.S. Cl. CPC C07K 16/2803; A61P 35/00; C07K 16/2809; A61K 38/00; A61K 2039/505; C07K 2317/31; C07K 2317/73; C07K 2317/92; C07K 2318/20; C07K 2319/31

(57) ABSTRACT

The present invention relates to recombinant binding proteins comprising an ankyrin repeat domain, wherein the ankyrin repeat domain has binding specificity for human CD33, in addition, the invention relates to nucleic acids encoding such recombinant binding proteins, pharmaceutical compositions comprising such proteins or nucleic acids, and the use of such binding proteins, nucleic acids or pharmaceutical compositions in methods for treating or diagnosing diseases, such as cancer, e.g., acute myeloid leukemia (AML), in mammal, including a human.

### CROSS-REFERENCE TO RELATED APPLICATIONS

The present application claims the benefit of priority to U.S. 63/158,539, filed on Mar. 9, 2021; U.S. 63/172,818, filed on Apr. 9, 2021; and U.S. 63/285,179, filed on Dec. 9, 2021. The disclosures of these patent applications are incorporated herein for all purposes by reference in their entirety.

### FIELD OF THE DISCLOSURE

The present invention relates to recombinant binding proteins comprising an ankyrin repeat domain, wherein the ankyrin repeat domain has binding specificity for human CD33. In addition, the invention relates to nudeic acids encoding such recombinant binding proteins, pharmaceutical compositions comprising such proteins or nucleic adds, and the use of such binding proteins, nucleic acids or pharmaceutical compositions in methods for treating or diagnosing diseases, such as cancer, e.g., acute myeloid leukemia (AML), In a mammal, Including a human.

### BACKGROUND

Acute myeloid leukemia (AML) is a heterogeneous and complex malignant disease characterized by rapid cellular proliferation, an aggressive clinical course and generally high mortality rates. Treatment resistance remains a leading cause of AML related deaths (Winer & Stone, Ther Adv Hematol; 2019; 10). While standard protocols employing chemotherapy are still the main therapeutic approach applied worldwide, recent advances in immunotherapy have provided effective treatment options for chemotherapy resistant AML. Such immunotherapy approaches include monoclonal antibodies, bispecific antibodies and chimeric antigen receptor-expressing T cells (CAR-T cells).

CD33 is an attractive target for the treatment of cancers, and particularly AML, as CD33 is expressed on approximately 80-90% of AML blast cells and leukemic stem cells (Ehninger et al., Blood Cancer Journal 4, e218, 2014). CD33 has also been clinically validated as a target for AML therapy, with anti-CD33 antibodies used either as monotherapy or conjugated with cytotoxic agents (Winer & Stone, Ther Adv Hematol 2019; 10). However, these drugs have shown either significant adverse effects or low efficacy. For example, treatment with gemtuzumab ozogamicin, a humanized, anti-CD33 monocdonal antibody conjugated to the cytotoxic agent calicheamicin, has resulted in significant hematologic and hepatic toxicity.

CAR-T cell therapy is an approach which has strongly affected the management of lymphoid malignancies. While there has been great interest in applying this technology also to AML, in practice this has proven challenging. As for monocdonal antibodies, CD33 has been considered among the most promising targets for CAR-T cell therapy in AML. However, pre-clinical models for this approach showed broad side effects on non-AML cells (on-target/off-tumor toxicity), and cytokine release syndrome (CRS) is another recognized side effect.

T cell-directed killing of tumor cells using bispecific antibodies is another recent therapeutic tool which has been utilized for the treatment of various cancer types, including AML. These T cel engager (TCE) bispecific antibodies comprise two different variable regions, one binding to the T cell receptor complex subunit CD3 and the other binding to a tumor cel surface antigen. Binding of a TCE to these two targets provides a functional connection between the cells, resulting in T cell activation and cytotoxic activity against the tumor cells, bypassing the normal TCR-MHC Interaction (Elerman, Methods; 154:102-117 (2019)). AMG330 is a bispecific antibody against CD3 and CD33 that can cause T cell cytotoxicity against AML cells.

However, both mono-specific and bi-specific antibody therapeutics can present various disadvantages, such as high production costs, and they can cause severe side effects, such as cytokine release syndrome (CRS) (Shimabukuro-Vornhagen et al, J Immunother Cancer; 6(1):56 (2018); Labrijn et al, Nat Rev Drug Discov; 18(8):585-608 (2019)) and/or on-target/off-tumor toxicities (Weiner et al, Cancer Res.; 55 (20): 4586-4593 (1995); Weiner et al, Cancer Immunol. Immunother.; 42 (3); 141-150 (1996)). Antibody-based T cell engagers often display more than 1000-fold higher affinity for CD3, as compared to the natural TCR-MHC Interaction (Wu at al, Phamacol Ther; 182:161-75 (2018); WO2014/167022; Junntila et al. Cancer Res.; 19:5561-71 (2014); Yang et al, J Immunol August; 15 (137):1097-100 (1986)). This high affinity has been correlated with lower efficiency of T cell activation and tumor cell killing (Bortoletto et al, Eur. J. Immunol. 32; 11: 3102-3107 (2002); Ellerman. Methods; 154:102-117(2019); Mandikian at al, Mol. Cancer Thor.; 17 (4): 776 LP-785 (2018); Vafa et al, Fronders in Oncology, 10: 446 (2020)). Furthermore, downregulation of the tumor surface marker targeted by the TCE can lead to resistance of the tumor to the TCE therapy.

Acute myeloid leukemia (AML) is a type of cancer that in many ways exemplifies the challenges for cancer therapy and the shortcomings of currently available cancer therapies, as discussed above. For AML, the medical need due to high mortality remains high, and the treatment of relapsed or refractory AML continues to be therapeutically challenging.

Thus, there remains a need for new CD33-specific binding proteins with beneficial properties. Such binding proteins may be useful for therapeutic and diagnostic approaches for the treatment and characterization of diseases, including cancer, such as AML. In particular, there is a need for new CD33-specific binding proteins that can serve to specifically target CD33 on cancer cells and that can also easily be combined with other functional moieties, such as, e.g., one or more binding moieties.

### SUMMARY

The present invention relates to recombinant binding proteins comprising an ankyrin repeat domain, wherein the ankyrin repeat domain has binding specificity for human CD33. In addition, the Invention relates to nucleic acids encoding such recombinant binding proteins, pharmaceutical compositions comprising such proteins or nucleic adds, and the use of such binding proteins, nucleic acids or pharmaceutical compositions in methods for treating or diagnosing diseases, such as cancer, e.g., acute myeloid leukemia (AML), in a mammal, including a human.

Recombinant binding proteins of the invention specifically bind to or target the tumor-associated antigen (TAA) CD33. Such binding proteins of the invention can serve as a tool or as a building block for the generation of new therapeutic or diagnostic agents. Also disclosed herein are recombinant binding proteins, in which the CD33-specific ankyrin repeat domains are combined with one or more other functional moieties in one molecule. Such other functional moieties include a binding moiety with binding specificity for a target expressed on an immune cell, a half-life extending moiety, a binding moiety with binding specificity for another tumor-associated antigen, and/or a cytotoxic agent. As such, recombinant binding proteins of the invention with binding specificity for CD33 are useful for the generation of novel therapeutic molecules, which may provide an improved toxicity profile and/or therapeutic window as compared to current therapeutic modalities.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

1. In a first embodiment, the invention relates to a recombinant binding protein comprising an ankyrin repeat domain, wherein said ankyrin repeat domain has binding specificity for human CD33, and wherein said ankyrin repeat domain comprises an ankyrin repeat module having an amino acid sequence selected from the group consisting of (1) SEQ ID NOs: 29 to 51 and 79 to 81, and (2) sequences in which up to 9 amino acids in any of SEQ ID NOs: 29 to 51 and 79 to 81 are substituted by another amino acid.

2. In a second embodiment, the invention relates to a recombinant binding protein comprising an ankyrin repeat domain, wherein said ankyrin repeat domain has binding specificity for human CD33, and wherein said ankyrin repeat domain comprises an amino acid sequence with at least 85% amino acid sequence identity with any one of SEQ ID NO: 1 to 16 and 77 to 78.

3. In a third embodiment, the invention relates to the recombinant binding protein of any preceding embodiment, wherein said ankyrin repeat domain binds human CD33 in PBS with a dissociation constant (K_{D}) below about 100 nM, optionally with a K_{D} between about 0.1 nM and about 100 nM.

4. In a fourth embodiment, the invention relates to the recombinant binding protein of any preceding embodiment, wherein said ankyrin repeat domain binds human CD33 with an EC₅₀ ranging from about from about 0.1 nM to about 10 nM.

5. In a fifth embodiment, the invention relates to the recombinant binding protein of any preceding embodiment, further comprising a binding moiety with binding specificity for a target expressed on an immune cell.

6. In a sixth embodiment, the invention relates to the recombinant binding protein of embodiment 5, wherein said immune cell Is a T cell and wherein said target expressed on an Immune cell is CD3.

7. In a seventh embodiment, the invention relates to the recombinant binding protein of any one of embodiments 5 to 6, wherein said binding moiety with binding specificity for a target expressed on an immune cell is an ankyrin repeat domain.

8. In an eighth embodiment, the invention relates to the recombinant binding protein of any of embodiments 5 to 7, wherein said binding moiety with binding specificity for a target expressed on an immune cell is an ankyrin repeat domain with binding specificity for human CD3.

9. In a ninth embodiment, the Invention relates to the recombinant binding protein of any of embodiment 5 to 7, wherein said binding moiety with binding specificity for a target expressed on an immune cell is an ankyrin repeat domain with binding specificity for human CD3, and wherein said ankyrin repeat domain with binding specificity for human CD3 comprises an amino acid sequence that is at least 85% amino acid sequence identity with any one of SEQ ID NOs: 55 to 59.

10. In a tenth embodiment, the invention relates to the recombinant binding protein of embodiment 9, wherein said ankyrin repeat domain with binding specificity for human CD3 comprises the amino acid sequence of any one of SEQ ID NOs: 55 to 59.

11. In an eleventh embodiment, the invention relates to the recombinant binding protein of any of embodiments 5 to 10, wherein said ankyrin repeat domain with binding specificity for human CD33 and said binding moiety with binding specificity for a target expressed on an immune cel are covalently linked with a peptide linker.

12. In a twelfth embodiment, the invention relates to the recombinant binding protein of embodiment 11, wherein said peptide linker is a proline-threonine-rich peptide linker.

13. In a thirteenth embodiment, the Invention relates to the recombinant binding protein of embodiments 11 to 12, wherein the amino acid sequence of said peptide linker has a length from 1 to 50 amino acids.

14. In a fourteenth embodiment, the invention relates to the recombinant binding protein of any preceding embodiment, wherein said binding protein further comprises a half-life extending moiety.

15. In a fifteenth embodiment, the invention relates to the recombinant binding protein of embodiment 14, wherein said half-life extending moiety is an ankyrin repeat domain with binding specificity for human serum albumin.

18. In a sixteenth embodiment, the invention relates to the recombinant binding protein of embodiment 15, wherein said ankyrin repeat domain with binding specificity for human serum albumin comprises an amino acid sequence that is at least 85% identical to the amino acid sequence of any one of SEQ ID NOs: 52 to 54.

17. In a seventeenth embodiment, the invention relates to the recombinant binding protein of embodiments 15 and 16, wherein said ankyrin repeat domain with binding specificity for human serum albumin comprises the amino acid sequence of any one of SEQ ID NOs: 52 to 54.

18. In an eighteenth embodiment, the invention relates to the recombinant binding protein of any of the preceding embodiments, wherein said binding protein further comprises at least one binding moiety with binding specificity for a target expressed in a tumor cell, wherein said target expressed in a tumor cel is different from human CD33.

19. In a nineteenth embodiment, the invention relates to a nucleic acid encoding the recombinant binding protein of any of the preceding embodiments.

20. In a twentieth embodiment, the invention relates to a pharmaceutical composition comprising the recombinant binding protein of any of embodiments 1 to 18 or the nucleic acid of embodiment 19, and a pharmaceutically acceptable carrier and/or diluent.

21. In a twenty first embodiment, the invention relates to a method of immune cell activation in a tumor tissue of a human patient, the method comprising the step of administering to said patient the recombinant binding protein of any one of embodiments 1 to 18, the nudeic acid of embodiment 19, or the pharmaceutical composition of embodiment 20.

22. In a twenty second embodiment, the invention relates to the method of embodiment 21, wherein said immune cell is a T cell.

23. In a twenty third embodiment, the invention relates to a method of treating a medical condition, the method comprising the step of administering to a patient in need thereof a therapeutically effective amount of the recombinant binding protein of any one of embodiments 1 to 18, the nucleic acid of embodiments 19, or the pharmaceutical composition of embodiments 20.

24. In a twenty fourth embodiment, the invention relates to the method of embodiment 23, wherein said medical condition is a cancer.

25. In a twenty fifth embodiment, the invention relates to the method of embodiment 23, wherein said medical condition is a cancer characterized by a liquid tumor.

28. In a twenty sixth embodiment, the invention relates to the method of embodiment 23, wherein said medical condition is leukemia.

27. In a twenty seventh embodiment, the invention relates to the method of embodiment 23, wherein said medical condition is acute myeloid leukemia.

28. In a twenty eighth embodiment, the invention relates to the recombinant binding protein of any one of embodiments 1 to 18, the nucleic acid of embodiment 19, or the pharmaceutical composition of embodiment 20, for use in therapy.

29. In a twenty ninth embodiment, the Invention relates to the recombinant binding protein of any one of embodiments 1 to 18, the nucleic acid of embodiment 19, or the pharmaceutical composition of embodiment 20, for use in treating cancer, optionally for use in treating a cancer characterized by a liquid tumor.

30. In a thirtieth embodiment, the invention relates to the recombinant binding protein or the pharmaceutical composition for use according to embodiment 29, wherein said cancer is leukemia, optionally wherein said cancer is acute myeloid leukemia.

### BRIEF DESCRIPTION OF THE DRAWINGS

FIG. 1 (A-B). Surface Plasmon Resonance (SPR) analysis of ankyrin repeat proteins binding to human CD33. FIG. 1A. SPR analysis for DARPin® protein #29; FIG. 1B. SPR analysis for DARPin® protein #30. Various concentrations of the purified ankyrin repeat protein were applied to a GLC chip with immobilized human CD33 for on-rate and off-rate measurements. The obtained SPR trace analyses were used to analyze and determine the binding of the ankyrin repeat proteins to CD33. RU, Resonance Units; s, time In seconds.

FIG. 2. Binding of exemplary binding proteins of the invention to CD33-expressing tumor cells. Shown are concentration-dependent binding curves of DARPin® protein #2, DARPin® protein #29 and DARPin® protein #30 (all in single domain format).

FIG. 3. Short term T cell activation determined by activation marker CD25. Pan-T effector cells and Molm-13 target cells were incubated at an E:T ratio of 5:1 and T-call activation assessed by FACS after 24 hours co-culture in the presence of serial dilutions of indicated molecules. Activated T-cells were gated as living CD8+/CD25+ cells. Shown Is the T cell activation induced by selected ankyrin repeat proteins DARPin® protein #33, DARPin® protein #31 and DARPin® protein #32.

FIG. 4. Tumor cell killing assessed by a cytotoxicity assay measuring LDH release. Pan-T effector cells and Molm-13 target cells were incubated at an E:T ratio of 5:1 and tumor cell killing was assessed by FACS after 24 hours co-culture in the presence of serial dilutions of indicated molecules. Shown is tumor cel killing by T cells triggered by DARPin® protein #33, DARPin® protein #31 and DARPin® protein #32.

FIG. 6. Binding of CD33 specific ankyrin repeat proteins to full-length and truncated CD33 target determined by HTRF. All three tested ankyrin repeat proteins, DARPin® protein #1, DARPin® protein #9 or DARPin® protein #14, bind to the full-length CD33, but only DARPin® protein #9 binds to the truncated CD33. Signals are shown in percentage (background corrected and normalized to maximal HTRF signal observed for each protein). HTRF signal in A.U. are shown as numbers.

FIG. 6. Binding Competition ELISA. Biotinylated human CD33 target was pre-incubated with or without competitor (DARPin® protein #1, DARPin® protein #9 or DARPin® protein #14); before binding target was tested against immobilized AMG330. Partial competition with AMG330 has been observed for DARPin® protein #1. DARPin® protein #14 shows full competition with AMG330 while DARPin® protein #9 shows no competition against AMG330

FIG. 7 (A-9): FIG. 7A. Tumor growth over time In mice Injected Intraperitoneally with hPBMC (n=5 mice per donor 12 hPBMC donors used), xenografted subcutaneously with MOLM-13 tumor cells two days after hPBMC injection, and treated with PBS 1× (black circle) or DARPin® protein #29 in a multi-domain format at 0.5 mg/kg (black square). Treatments were started at day 4 after tumor cell xenograft. Data are presented in average+SEM. FIG. 7B. Evaluation of tumor volume at day 17 after tumor cell xenograft in the mice described in FIG. 7A.

FIG. 8 (A-B). FIG. 8A. Potency titration curves (T cell activation) of DARPin® protein #29 in a multi-domain format using wildtype or CD33 knockout Molm-13 tumor cells. EC50 values are shown in μM. FIG. 8B. Potency titration curves (T cell activation) of DARPin® protein #30 in a multi-domain format using wildtype or CD33 knockout Molm-13 tumor cells. EC50 values ae shown in pM.

### DETAILED DESCRIPTION OF THE INVENTION

Disclosed herein are recombinant binding proteins comprising an ankyrin repeat domain, wherein said ankyrin repeat domain has binding specificity for human CD33. Also disclosed are nucleic acids encoding the recombinant binding proteins, pharmaceutical compositions comprising the binding proteins or nucleic acids, and methods of using the binding proteins, nucleic acids, or pharmaceutical compositions.

### Ankyrin Repeat Domains

The described recombinant binding proteins, or binding domains thereof, comprising designed ankyrin repeat motifs or modules are also referred herein as DARPin® proteins (see Stumpp et al., Curr Opin Drug Discov Devel. 10(2): 153-9 (2007); and Binz et al., Nature Biotech. 22(5): 575-582 (2004)). DARPin® proteins can be considered as antibody mimetics with high specificity and high binding affinity to a target protein. In general, a DARPin® protein comprises at least one ankyrin repeat domain, and may comprise 2, 3, 4, 5, or more ankyrin repeat domains.

The ankyrin repeat domains described herein generally comprise a core scaffold that provides structure, and target binding residues that bind to a target. The structural core includes conserved amino acid residues, and the target binding surface includes amino acid residues that differ depending on the target.

Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats.

Designed ankyrin repeat proteins may also target epitopes which are not readily accessible with monoclonal antibodies. Further advantages of the described designed ankyrin repeat proteins are that they generally have low immunogenic potential and no or insignificant off-target effects. DARPin® candidates also display favorable development properties including rapid, low-cost and high-yield manufacturing and up to several years of shelf-life at 4° C. Taken together, designed ankyrin repeat proteins are an example of the next generation of protein therapeutics with the potential to surpass existing antibody drugs.

DARPin® is a trademark owned by Molecular Partners AG, Switzerland.

As discussed above, CD33 is an attractive therapeutic target for the treatment of certain cancers, particularly AML. The recombinant binding proteins described herein comprise an ankyrin repeat domain and ankyrin repeat modules that specifically bind to human CD33.

In one embodiment, the present Invention relates to a recombinant binding protein comprising an ankyrin repeat domain, wherein said ankyrin repeat domain has binding specificity for human CD33, and wherein said ankyrin repeat domain comprises an ankyrin repeat module having an amino acid sequence selected from the group consisting of (1) SEQ ID NOs: 29 to 51 and 79 to 81, and (2) sequences in which up to 9 amino acids in any of SEQ ID NOs: 29 to 51 and 79 to 81 are substituted by another amino acid.

In one embodiment, the present invention relates to a recombinant binding protein comprising an ankyrin repeat domain, wherein said ankyrin repeat domain has binding specificity for human CD33, and wherein said ankyrin repeat domain comprises an ankyrin repeat module having an amino acid sequence selected from the group consisting of (1) SEQ ID NOs: 29 to 51 and 79 to 81, and (2) sequences in which up to 9, up to 8, up to 7, up to 6, up to 5, up to 4, up to 3, up to 2, or up to 1 amino acids in any of SEQ ID NO.: 29 to 51 and 79 to 81 are substituted by another amino acid.

In one embodiment, the present invention relates to a recombinant binding protein comprising an ankyrin repeat domain, wherein said ankyrin repeat domain has binding specificity for human CD33, and wherein said ankyrin repeat domain comprises an ankyrin repeat module having an amino acid sequence selected from the group consisting of SEQ ID NOs: 29 to 51 and 79 to 81.

In another embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity with any one of SEQ ID NOs: 1 to 16 and 77 to 78.

In a further embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity with any one of SEQ ID NOs: 1 to 16 and 77 to 78.

In one embodiment, the present Invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence Identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 1.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence Identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 2.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 3.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 4.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 5.

In one embodiment, the present Invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 6.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 7.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 8.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 9.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 10.

In one embodiment, the present Invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence Identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 11.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence Identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 12.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 13.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 14.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 15.

In one embodiment, the present Invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 16.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 77.

In one embodiment, the present invention relates to a recombinant binding protein comprising an ankyrin repeat domain, wherein said ankyrin repeat domain has binding specificity for human CD33, and wherein said ankyrin repeat domain comprises an amino acid sequence with at least about 85% amino acid sequence Identity, such as at least about 88%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with SEQ ID NO: 78.

In a further embodiment, the present invention relates to a recombinant binding protein comprising an ankyrin repeat domain, wherein said ankyrin repeat domain has binding specificity for human CD33, and wherein said ankyrin repeat domain comprises an amino acid sequence selected from the group consisting of SEQ ID NOs: 1 to 16 and 77 to 78.

In a further aspect, the invention relates to the recombinant binding protein as described above, further comprising at least one binding moiety with binding specificity for a target expressed on an immune cell.

In one embodiment, said Immune cell is a T cell. In another embodiment, said Immune cell is a Natural Killer (NK) cell. Examples of binding moieties with binding specificity for a target expressed on an immune cell for use in the present invention include antibodies, alternative scaffolds, and polypeptides.

Antibodies include any polypeptides or proteins comprising an antigen binding domain that is derived from an antibody or immunoglobulin molecule. The antigen binding domain can be derived, for example, from monoclonal antibodies, polyclonal antibodies, recombinant antibodies, human antibodies, humanized antibodies, and single-domain antibodies, e.g., a heavy chain variable domain (VH), a light chain variable domain (VL) and a variable domain (VHH) from, e.g., human or camelid origin. In some instances, it is beneficial for the antigen binding domain to be derived from the same species in which the binding moiety win ultimately be used in. For example, for use in humans, it may be beneficial for the antigen binding domain of the binding moiety described herein, to comprise a human or a humanized antigen binding domain. Antibodies can be obtained using techniques well known in the art.

In one embodiment, the binding moiety with binding specificity for a target expressed on an immune cel is an antibody.

In one embodiment, the binding moiety with binding specificity for a target expressed on an immune cell is a camelid nanobody. Camelid nanobodies (also known as camelid single-domain antibodies or VHHs) are derived from the Camelidae family of mammals such the llamas, camels, and alpacas. Unlike other antibodies, camelid antibodies lack a fight chain and are composed of two identical heavy chains. Camelid antibodies typically have a relatively low molecular weight in the region of around 15 kDa.

In one embodiment, the binding moiety with binding specificity for a target expressed on an immune cel is a shark antibody domain. Shark antibody domains, like camelid nanobodies, also lack a light chain.

Alternative scaffolds include any polypeptides or proteins comprising a binding domain that is capable of binding an antigen (such as a drug molecule) and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin® proteins), fynomers, knottins, and Kunitz domain peptides. Alternative scaffolds are described, e.g., in Yu et al., Annu Rev Anal Chem (Palo Alto Calif.). 2017 Jun. 12; 10(1): 293-320. doi:10.1146/annurevanchem-061516-045205.

In one embodiment, the binding moiety with binding specificity for a target expressed on an immune cel is an alternative scaffold. In one embodiment, the binding moiety with binding specificity for a target expressed on an immune cell comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cel receptor (TCR).

Adnectins are originally derived from the tenth extracellular domain of human fibronectin type Ill protein (10Fn3). The fibronectin type II domain has 7 or 8 beta strands, which are distributed between two beta sheets, which themselves pack against each other to form the core of the protein, and further contain loops (analogous to CDRs), which connect the beta strands to each other and are solvent exposed.

There are at least three such loops at each edge of the beta sheet sandwich, where the edge is the boundary of the protein perpendicular to the direction of the beta strands (see U.S. Pat. No. 6,818,418).

Because of this structure, this non-antibody scaffold mimics antigen binding properties that are similar in nature and affinity to those of antibodies. These scaffolds can be used in a loop randomization and shuffling strategy in vitro that is similar to the process of affinity maturation of antibodies in vivo.

Affibody affinity ligands are composed of a three-helix bundle based on the scaffold of one of the IgG-binding domains of Protein A, which is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, but are considerably smaller, having a molecular weight of around 6 kDa, compared to around 150 kDa for antibodies. Despite the size difference, the binding site of affibody molecules has similarity to that of an antibody.

Affilins are synthetic antibody mimetics that are structurally derived from human ubiquitin (historically also from gamma-B crystallin). Affilins consists of two identical domains with mainly beta sheet structure and a total molecular mass of about 20 kDa. They contain several surface-exposed amino acids that are suitable for modification. Affilins resemble antibodies in their affinity and specificity to antigens but not in structure.

Affimers are a type of peptide aptamer, having a structure known as SQT (Stefin A quadruple mutant-Tracy). Aptamers and affimers are short peptides responsible for affinity binding with an inert and rigid protein scaffold for structure constraining in which both N- and C-termini of the binding peptide are embedded in the inert scaffold.

Affitins are variants of the DNA binding protein Sac7d that are engineered to obtain specific binding affinities. Sac7d is originally derived from the hyperthermophile archaea Sulfolobus acidocaldarius and binds with DNA to prevent it from thermal denaturation. Affitins are commercially known as Nanofitins.

Alphabodies are small (approximately 10 kDa) proteins that are engineered to bind to a variety of antigens and are therefore antibody mimetics. The alphabody scaffold is computationally designed based on coiled-coil structures. The standard alphabody scaffold contains three α-helices, composed of four heptad repeats (stretches of 7 residues) each, connected via glycine/serine-rich linkers. The standard heptad sequence Is “IAAIQKQ”. Alphabodies' ability to target extracellular and intracellular proteins in combination with their high binding affinities may allow them to bind to targets that cannot be reached with antibodies.

Anticalins are a group of binding proteins with a robust and conservative β-barrel structure found in lipocalins. Lipocalins are a class of extracellular proteins comprising one peptide chain (150-190 amino acids) that is in charge of recognition, storage, and transport of various biological molecules such as signaling molecules.

Armadillo repeat protein-based scaffolds are abundant in eukaryotes and are involved In a broad range of biological processes, especially those related to nuclear transport. Armadillo repeat protein-based scaffolds usually consist of three to five internal repeats and two capping elements. They also have a tandem elongated super helical structure that enables binding with their corresponding peptide ligands in an extended conformation.

Atrimers are a scaffold derived from a trimeric plasma protein known as tetranectin, belonging to a family of C-type lectins consisting of three identical units. The structure of the C-type lectin domain (CTLD) within the tetranectin has five flexible loops that mediate interaction with targeting molecules.

Avimers are derived from natural A-domain containing proteins such as HER3 and consist of a number of different “A-domain” monomers (2-10)linked via amino acid linkers. Avimers can be created that can bind to the target antigen using the methodology described in, for example, U.S. Patent Application Publication Nos. 2004/0175756; 2005/0053973; 2005/0048512; and 2006/0008844.

Fynomers are small globular proteins (approximately 7 kDa) that evolved from amino acids 83-145 of the Src homology domain 3 (SH3) of the human Fyn tyrosine kinase. Fynomers are attractive binding molecules due to their high thermal stability, cysteine-free scaffold, and human origin, which reduce potential immunogenicity.

Knottins, also known as cysteine knot miniproteins, are typically proteins 30 amino acids in length comprising three antiparallel β-sheets and constrained loops laced by a disulfide bond, which creates a cysteine knot. This disulfide bond confers high thermal stability making knottins attractive antibody mimetics.

Kunitz domain peptides or Kunitz domain inhibitors are a class of protease inhibitors with irregular secondary structures containing ˜60 amino acids with three disulfide bonds and three loops that can be mutated without destabilizing the structural framework.

In one embodiment, the binding moiety with binding specificity for a target expressed on an immune cell is a polypeptide or protein comprising an antigen binding domain derived from a T cell receptor (TCR).

In a preferred embodiment, the binding moiety with binding specificity for a target expressed on an immune cell is an ankyrin repeat domain.

There is no particular limit on the nature of the target expressed on said immune cell. In one embodiment, the target is expressed on an immune cell that is a T cell and the target expressed on said immune cell is CD3.

Thus, in a preferred embodiment, the invention relates to a recombinant binding protein comprising (i) a first ankyrin repeat domain, wherein said first ankyrin repeat domain has binding specificity for human CD33, and wherein said first ankyrin repeat domain comprises an ankyrin repeat module having an amino acid sequence selected from the group consisting of (1) SEQ ID NOs: 29 to 51 and 79 to 81, and (2) sequences in which up to 9, up to 8, up to 7, up to 6, up to 5, up to 4, up to 3, up to 2 or up to 1 amino acids in any of SEQ ID NOs: 29 to 51 and 79 to 81 are substituted by another amino acid, and (ii) a second ankyrin repeat domain, wherein said second ankyrin repeat domain has binding specificity for CD3, more preferably human CD3.

In another embodiment, the invention relates to a recombinant binding protein comprising (i) a first ankyrin repeat domain, wherein said first ankyrin repeat domain has binding specificity for human CD33, and wherein said first ankyrin repeat domain comprises an ankyrin repeat module having an amino acid sequence selected from the group consisting of (1) SEQ ID NOs: 29 to 51 and 79 to 81, and (2) sequences in which up to 9, up to 8, up to 7, up to 8, up to 5, up to 4, up to 3, up to 2 or up to 1 amino acids in any of SEQ ID NOs: 29 to 51 and 79 to 81 are substituted by another amino acid, and (ii) a second ankyrin repeat domain, wherein said second ankyrin repeat domain has binding specificity for human CD3, and wherein said second ankyrin repeat domain comprises an amino acid sequence with at least about 85% amino acid sequence Identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with any one of SEQ ID NOs: 55 to 59.

In a further embodiment, the invention relates to a recombinant binding protein comprising (i) a first ankyrin repeat domain, wherein said first ankyrin repeat domain has binding specificity for human CD33, and wherein said first ankyrin repeat domain comprises an ankyrin repeat module having an amino acid sequence selected from the group consisting of SEQ ID NOs: 29 to 51 and 79 to 81, and (ii) a second ankyrin repeat domain, wherein said second ankyrin repeat domain has binding specificity for human CD3, and wherein said second ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with any one of SEQ ID NOs: 55 to 59.

In another embodiment, the invention relates to a recombinant binding protein comprising (i) a first ankyrin repeat domain, wherein said first ankyrin repeat domain has binding specificity for human CD33, and wherein said first ankyrin repeat domain comprises an ankyrin repeat module having an amino acid sequence selected from the group consisting of (1) SEQ ID NOs: 29 to 51 and 79 to 81, and (2) sequences in which up to 9, up to 8, up to 7, up to 6, up to 5, up to 4, up to 3, up to 2 or up to 1 amino acids In any of SEQ ID NOs: 29 to 51 are substituted by another amino acid, and (0) a second ankyrin repeat domain, wherein said second ankyrin repeat domain has binding specificity for human CD3, and wherein said second ankyrin repeat domain comprises the amino acid sequence of any one of SEQ ID NOs: 55 to 59.

In another embodiment, the invention relates to a recombinant binding protein comprising (i) a first ankyrin repeat domain, wherein said first ankyrin repeat domain has binding specificity for human CD33, and wherein said first ankyrin repeat domain comprises an ankyrin repeat module having an amino acid sequence selected from the group consisting of SEQ ID NOs: 29 to 51 and 79 to 81, and (ii) a second ankyrin repeat domain, wherein said second ankyrin repeat domain has binding specificity for human CD3, and wherein said second ankyrin repeat domain comprises the amino acid sequence of any one of SEQ ID NOs: 55 to 59.

In another preferred embodiment, the invention relates to a recombinant binding protein comprising (i) a first ankyrin repeat domain, wherein said first ankyrin repeat domain has binding specificity for human CD33, and wherein said first ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity with any one of SEQ ID NOs: 1 to 16 and 77 to 78, and (ii) a second ankyrin repeat domain, wherein said second ankyrin repeat domain has binding specificity for CD3, more preferably human CD3.

In another embodiment, the invention relates to a recombinant binding protein comprising (i) a first ankyrin repeat domain, wherein said first ankyrin repeat domain has binding specificity for human CD33, and wherein said first ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with any one of SEQ ID NOs: 1 to 16 and 77 to 78, and (ii) a second ankyrin repeat domain, wherein said second ankyrin repeat domain has binding specificity for human CD3, and wherein said second ankyrin repeat domain comprises an amino acid sequence with at least about 85% sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with any one of SEQ ID NOs: 55 to 59.

In one embodiment, the invention relates to a recombinant binding protein comprising (i) a first ankyrin repeat domain, wherein said first ankyrin repeat domain has binding specificity for human CD33, and wherein said first ankyrin repeat domain comprises the amino acid sequence of any one of SEQ ID NOs: 1 to 16 and 77 to 78, and (ii) a second ankyrin repeat domain, wherein said second ankyrin repeat domain has binding specificity for human CD3, and wherein said second ankyrin repeat domain comprises an amino acid sequence with at least about 85% sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with any one of SEQ ID NOs: 55 to 59.

In one embodiment, the invention relates to a recombinant binding protein comprising (i) a first ankyrin repeat domain, wherein said first ankyrin repeat domain has binding specificity for human CD33, and wherein said first ankyrin repeat domain comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence identity, with any one of SEQ ID NOs: 1 to 16 and 77 to 78, and (ii) a second ankyrin repeat domain, wherein said second ankyrin repeat domain has binding specificity for human CD3, and wherein said second ankyrin repeat domain comprises the amino acid sequence of any one of SEQ ID NOs: 55 to 59.

In a further embodiment, the invention relates to a recombinant binding protein comprising (i) a first ankyrin repeat domain, wherein said first ankyrin repeat domain has binding specificity for human CD33, and wherein said first ankyrin repeat domain comprises the amino acid sequence of any one of SEQ ID NOs:1 to 16 and 77 to 78, and (ii) a second ankyrin repeat domain, wherein said second ankyrin repeat domain has binding specificity for human CD3, and wherein said second ankyrin repeat domain comprises the amino acid sequence of any one of SEQ ID NOs: 55 to 59.

The invention further relates to a recombinant binding protein comprising (i) a first ankyrin repeat domain, wherein said first ankyrin repeat domain has binding specificity for human CD33, and (ii) a second ankyrin repeat domain, wherein said second ankyrin repeat domain has binding specificity for human CD3, wherein said recombinant binding protein comprises an amino acid sequence with at least about 85% amino acid sequence identity, such as at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% amino acid sequence Identity, with any one of SEQ ID NOs: 17 to 28 and 82 to 84.

In one embodiment, the invention relates to a recombinant binding protein comprising (i) a first ankyrin repeat domain, wherein said first ankyrin repeat domain has binding specificity for human CD33, and (ii) a second ankyrin repeat domain, wherein said second ankyrin repeat domain has binding specificity for human CD3, wherein said recombinant binding protein comprises an amino acid sequence with at least about 85% amino acid sequence Identity with any one of SEQ ID NOs: 17 to 28 and 82 to 84.

### Half-Life Extending Moieties

A “half-life extending moiety” extends the serum half-life in vivo of the recombinant binding proteins described herein, compared to the same protein without the half-life extending moiety. Examples of half-life extending moieties include, but are not limited to, polyhistidine, Glu-Glu, glutathione S transferase (GST), thioredoxin, protein A, protein G, an immunoglobulin domain, maltose binding protein (MBP), a human serum albumin (HSA) binding domain, or polyethylene glycol (PEG).

In some embodiments, the recombinant binding proteins described herein comprise an ankyrin repeat domain that specifically binds serum albumin (such as preferably human serum albumin), also referred herein as “serum albumin binding domain”. The recombinant binding protein described herein may also comprise more than one serum albumin binding domain, for example, two or three serum albumin binding domains. Thus, the recombinant binding protein described herein may comprise a first and a second serum albumin binding domain, or a first, a second and a third serum albumin binding domain. The embodiments provided below describe such a first serum albumin binding domain, second serum albumin binding domain, and/or third serum albumin binding domain.

In some embodiments, the half-life extending moiety described herein comprises a serum albumin-specific ankyrin repeat domain comprising an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 52 to 54. In an exemplary embodiment, the half-life extending moiety described herein comprises an amino acid sequence that is at least about 90% identical to any one of SEQ ID NOs: 52 to 54. In one embodiment, the half-life extending moiety described herein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 53. In an exemplary embodiment, the half-life extending moiety described herein comprises an amino acid sequence that is at least 90% identical to SEQ ID NO: 53.

In some embodiments, a serum albumin binding domain is located at the N-terminus of the recombinant binding protein of the invention. In some embodiments, two or more serum albumin binding domains are preferred. In some embodiments, two serum albumin binding domains are located at the N-terminus of the recombinant binding protein of the invention.

In some embodiments, the half-life extending moiety comprises an immunoglobulin domain. In some embodiments, the immunoglobulin domain comprises an Fc domain. In some embodiments, the Fc domain is derived from any one of the known heavy chain isotypes: IgG (γ), IgM (μ), IgD (δ), IgE (ε), or IgA (α). In some embodiments, the Fc domain is derived from any one of the known heavy chain isotypes or subtypes: IgG₁ (γ1), IgG₂(γ₂), IgG₃(γ3), IgG₄ (γ4), IgA₁ (α1), IgA₂(α2). In some embodiments, the Fc domain is the Fc domain of human IgG₁.

In some embodiments, the Fc domain comprises an uninterrupted native sequence (i.e., wild type sequence) of an Fc domain. In some embodiments, the immunoglobulin Fc domain comprises a variant Fc domain resulting in altered biological activity. For example, at least one point mutation or deletion may be introduced into the Fc domain so as to reduce or eliminate the effector activity (e.g., International Patent Publication No. WO 2005/063815), and/or to increase the homogeneity during the production of the recombinant binding protein. In some embodiments, the Fc domain Is the Fc domain of human IgG₁ and comprises one or more of the following effector-null substitutions: L234A, L235A, and G237A (Eu numbering). In some embodiments, the Fc domain does not comprise the lysine located at the C-terminal position of human IgG1 (i.e., K447 by Eu numbering). The absence of the lysine may increase homogeneity during the production of the recombinant binding protein. In some embodiments, the Fc domain comprises the lysine located at the C-terminal position (K447, Eu numbering).

Further Binding Moieties with Binding Specificity for a Tumor-Associated Antigen

In a further aspect, the invention relates to the recombinant binding protein as described above, further comprising at least one binding moiety with binding specificity for a tumor-associated antigen (TAA) that is different from CD33. In one embodiment, said one or more TAA that is different from CD33 is a TAA that is co-expressed with CD33 in cells from the same cancer. In a further embodiment, said one or more TAA that is different from CD33 is a TAA that is co-expressed with CD33 in a cancer characterized by a liquid tumor. In a preferred embodiment, said one or more TAA that is different from CD33 is a TAA that is co-expressed with CD33 in a leukemia, such as a TAA co-expressed with CD33 in AML cancer cells. Examples of binding moieties with binding specificity for a tumor-associated antigen (TAA) that is different from CD33 for use in the present invention include antibodies, alternative scaffolds, and polypeptides. Many TAAs are known in the art, including TAAs that are expressed In AML cancer cells.

### Substitutions

In some embodiments, no more 9, no more than 8, no more than 7, no more than 6, no more than 5, no more than 4, no more than 3, no more than 2, or no more than 1 substitution is made in any ankyrin repeat module of a recombinant binding protein of the invention relative to the sequences of SEQ ID NOs: 29 to 51 and 79 to 81. In some embodiments, no more than 5 substitutions are made relative to the sequences of SEQ ID NOs: 29 to 51 and 79 to 81. In some embodiments, no more than 4 substitutions are made relative to the sequences of SEQ ID NOs: 29 to 51 and 79 to 81. In some embodiments, no more than 3 substitutions are made relative to the sequences of SEQ ID NOs: 29 to 51 and 79 to 81. In some embodiments, no more than 2 substitutions are made relative to the sequences of SEQ ID NOs: 29 to 51 and 79 to 81. In some embodiments, no more than 1 substitution is made relative to the sequences of SEQ ID NOs: 29 to 51 and 79 to 81.

In some embodiments, no more 15%, no more than 14%, no more than 13%, no more than 12%, no more than 11%, no more than 10%, no more than 9%, no more than 8%, or no more than 7%, no more than 6%, no more than 5%, no more than 4%, no more than 3%, no more than 2%, or no more than 1% of the amino acid sequence of any ankyrin repeat domain of a recombinant binding protein of the invention is altered by substitutions relative to the sequences of SEQ ID NOs: 1 to 16 and 77 to 78. In some embodiments, no more 10% of the amino acid sequence is altered by substitutions relative to the sequences of SEQ ID NOs: 1 to 16 and 77 to 78. In some embodiments, no more 8% of the amino acid sequence is altered by substitutions relative to the sequences of SEQ ID NOs:1 to 16 and 77 to 78. In some embodiments, no more 6% of the amino acid sequence is altered by substitutions relative to the sequences of SEQ ID NOs: 1 to 16 and 77 to 78. In some embodiments, no more 4% of the amino acid sequence is altered by substitutions relative to the sequences of SEQ ID NOs: 1 to 16 and 77 to 78. In some embodiments, no more 2% of the amino acid sequence is altered by substitutions relative to the sequences of SEQ ID NOs: 1 to 16 and 77 to 78.

In some aspects, amino acid substitution(s) made to the binding agents do not change the K_{D} value by more than about 1000-fold, more than about 100-fold, or more than about 10-fold, compared to the K_{D} value of the unsubstituted binding agents. For example, in some aspects, the amino acid substitution(s) do not change the K_{D} value by more than about 1000-fold, more than about 300-fold, more than about 100-fold, more than about 50-fold, more than about 25-fold, more than about 10-fold, or more than about 5-fold, compared to the K_{D} value of the binding agent comprising any of the sequences of SEQ ID NOs:1 to 16, 29 to 51, 77 to 81 to CD33.

In certain embodiments, the substitution is a conservative substitution according to Table 1. In certain embodiments, the substitution is made outside the structural core residues of the ankyrin repeat domain, e.g., in the beta loops that connect the alpha-helices.

TABLE 1
Amino Acid Substitutions

| | Conservative | |
|---|---|---|
| Original Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | Ile; Leu; Met; Phe; Ala; Norleucine |

In certain embodiments, the substitution is made within the structural core residues of the ankyrin repeat domain. In other embodiments, the substitution is made outside the structural core residues of the ankyrin repeat domain. For illustration, the ankyrin domain may comprise the consensus sequence: xDxxGxTPLHLAxxxGxxxIVxVLLxxGADVNA (SEQ ID NO: 62), wherein “x” denotes any amino acid (preferably not cysteine, glycine, or proline); or xDxxGxTPLHLAAxxGHLEIVEVLLKzGADVNA (SEQ ID NO: 63), wherein “x” denotes any amino acid (preferably not cysteine, glycine, or proline), and “z” is selected from the group consisting of asparagine, histidine, or tyrosine. In one embodiment, the substitution is made to residues designated as “x”. In another embodiment, the substitution is made to residues that are not designated as “x”.

In addition, the second last position of any ankyrin repeat domain of a recombinant binding protein of the Invention can be “A” or “L”, and/or the last position can be “A” or “N”. Accordingly, in some embodiments, each ankyrin repeat domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 16, 52 to 59 and 77 to 78, and wherein optionally A at the second last position is substituted with L and/or A at the last position is substituted with N. or wherein optionally L at the second last position is substituted with A and/or N at the last position is substituted with A. In an exemplary embodiment, each ankyrin repeat domain comprises an amino acid sequence that is at least 90% identical to any one of SEQ ID NOs: 1 to 16, 52 to 58 and 77 to 78, and wherein optionally A at the second last position Is substituted with L and/or A at the last position is substituted with N. Furthermore, the sequence of any ankyrin repeat domain comprised in a binding protein of the invention may optionally comprise at its N-terminus, a G, an S, or a GS (see below).

In addition, each ankyrin repeat domain comprised In a recombinant binding protein of the invention may optionally comprise a “G,” an “S,” or a “GS” sequence at its N-terminus. Accordingly, in some embodiments, each ankyrin repeat domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 16, 52 to 59 and 77 to 78, and further comprises at its N-terminus a GS (as e.g. in SEQ ID NO: 64) or only a G or an S instead of the GS.

### Binding Affinity

In certain embodiments, the affinity between the recombinant binding protein and its target (i.e., human CD33) is described in terms of K_{D}. In exemplary embodiments, the K_{D} is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, from about 10⁻¹⁰ M to about 10⁻¹³ M, from about 10⁻⁵ M to about 10⁻¹² M, from about 10⁻⁶ M to about 10⁻¹² M, from about 10⁻⁷ M to about 10⁻¹² M, from about 10⁻⁸ M to about 10⁻¹² M, from about 10⁻⁹ M to about 10⁻¹² M, from about 10⁻¹⁰ M to about 10⁻¹² M, from about 10⁻⁵ M to about 10⁻¹¹ M, from about 10⁻⁶ M to about 10⁻¹¹ M, from about 10⁻⁷ M to about 10⁻¹¹ M, from about 10⁻⁵ M to about 10⁻¹¹ M, from about 10⁻⁹ M to about 10⁻¹¹ M, from about 10⁻¹⁰ M to about 10⁻¹¹ M, from about 10⁻⁵ M to about 10⁻¹⁰ M, from about 10⁻⁶ M to about 10⁻¹⁰ M, from about 10⁻⁷ M to about 10⁻¹⁰ M, from about 10⁻⁸ M to about 10⁻¹⁰ M, from about 10⁻⁹ M to about 10⁻¹⁰ M, from about 10⁻⁵ M to about 10⁻⁹ M, from about 10⁻⁶ M to about 10⁻⁹ M, from about 10⁻⁹ M to about 10⁻⁹ M, or from about 10⁻⁸ M to about 10⁻⁹ M.

In exemplary embodiments, the recombinant binding protein binds human CD33 with an Ku value of, or less than: about 900 nM, about 800 nM, about 700 nM, about 600 nM, about 500 nM, about 400 nM, about 300 nM, about 250 nM, about 200 nM, about 150 nM, about 100 nM, about 50 nM, about 40 nM, about 30 nM, about 20 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 10 pM, or about 1 pM. In one exemplary embodiment, the recombinant binding protein binds CD33 with a K_{D} value of less than or equal to 100 nM. In another exemplary embodiment, the recombinant binding protein binds CD33 with a K_{D} value of less than or equal to 10 nM In one aspect, the recombinant binding protein binds human CD33 with an EC₅₀ of less than about 5000, about 4000, about 3000, about 2000, about 1000, about 900, about 700, about 500, about 400, about 300, about 200, about 150, about 100, about 70, about 60, about 50, about 40, about 30, about 20, about 15, about 10, about 7, about 5, about 3, about 1, about 0.5, or about 0.1 nM. Thus, in one aspect, said binding protein binds human CD33 on T cells with an EC₅₀ of less than about 5 μM; in another aspect, said binding protein binds human CD33 on T cells with an EC₅₀ of less than about 4 μM; in another aspect, said binding protein binds human CD33 on T cells with an EC₅₀ of less than about 3 pM; in another aspect, said binding protein binds human CD33 on T cells with an EC₅₀ of less than about 2 μM; in another aspect, said binding protein binds human CD33 on T cells with an EC₅₀ of less than about 1 μM; in another aspect, said binding protein binds human CD33 on T cells with an ECs of less than about 900 nM; in another aspect, said binding protein binds human CD33 on T cells with an EC₅₀ of less than about 800 nM; in another aspect, said binding protein binds human CD33 on T cells with an EC₅₀ of less than about 600 nM in another aspect, said binding protein binds human CD33 on T cells with an EC₅₀ of less than about 700 nM; in another aspect, said binding protein binds human CD33 on T cells with an EC₅₀ of less than about 500 nM; in another aspect, said binding protein binds human CD33 on T cells with an EC₅₀ of less than about 400 nM; in another aspect, said binding protein binds human CD33 on T cells with an EC₅₀ of less than about 300 nM; in another aspect, said binding protein binds human CD33 on T cells with an EC₅₀ of less than about 200 nM; in another aspect, said binding protein binds human CD33 on T cells with an EC₅₀ of less than about 100 nM; in another aspect, said binding protein binds human CD33 on T cells with an EC₅₀ of less than about 70 nM; in another aspect, said binding protein binds human CD33 on T cells with an EC₅₀ of less than about 60 nM; in another aspect, said binding protein binds human CD33 on T cells with an EC₅₀ of less than about 50 nM; in another aspect, said binding protein binds human CD33 on T cells with an EC₅₀ of less than about 40 nM; in another aspect, said binding protein binds to CD33 with an EC₅₀ of less than about 30 nM; in another aspect, said binding protein binds to CD33 with an EC₅₀ of less than about 20 nM; In another aspect, said binding protein binds to CD33 with an EC₅₀ of less than about 15 nM; in another aspect, said binding protein binds to CD33 with an EC₅₀ of less than about 10 nM; in another aspect, said binding protein binds to CD33 with an EC₅₀ of less than about 7 nM; in another aspect, said binding protein binds to CD33 with an EC₅₀ of less than about 5 nM; in another aspect, said binding protein binds to CD33 with an EC₅₀ of less than about 3 nM; In another aspect, said binding protein binds to CD33 with an EC₅₀ of less than about 1 nM; in another aspect, said binding protein binds to CD33 with an EC₅₀ of less than about 0.5 nM; in a further aspect, said binding protein binds to CD33 with an EC₅₀ of less than about 0.1 nM.

### Additional Polypeptides

In one aspect, the recombinant binding protein of the invention further comprises a polypeptide tag. A polypeptide tag is an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags of a recombinant binding protein may be connected to other parts of the recombinant binding protein directly or via a peptide Inker. Polypeptide tags are all well known in the art and are fully available to the person skilled in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules.

In one aspect, the recombinant binding protein of the invention further comprises a peptide linker. A peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a localizer, or two sequence tags. Peptide linkers are known to the person skilled in the art. A list of examples is provided in the description of patent application WO2002/020565. In one aspect, peptide linkers for use in the present invention have a length from 1 to 50 amino acids. In another aspect, peptide linkers for use In the present Invention have a length from about 5 to about 40 amino acids. In another aspect, peptide linkers for use in the present Invention have a length from about 10 to about 30 amino adds.

Particular examples of peptide linkers are glycine-serine-linkers and proline-threonine rich linkers of variable lengths. In the context of the present invention a proline-threonine rich linker comprises at least about 20% proline residues and at least about 20% threonine residues in its amino acid sequence. Examples of a glycine-serine-linker are the amino acid sequence GS and the amino acid sequence of SEQ ID NO: 67, and examples of a proline-threonine rich linker are the amino acid sequences of SEQ ID NOs: 65 and 66.

### N-Terminal and C-Terminal Capping Sequences

The ankyrin repeat domains of the recombinant binding protein disclosed herein may comprise N-terminal or C-terminal capping sequences. Capping sequences refers to additional polypeptide sequences fused to the N- or C-terminal end of the ankyrin repeat sequence motif(s), wherein said capping sequences form tight tertiary interactions (i.e., tertiary structure interactions) with the ankyrin repeat sequence motif(s), thereby providing a cap that shields the hydrophobic core of the ankyrin repeat domain at the side from exposing to the solvent.

The N- and/or C-terminal capping sequences may be derived from, a capping unit or other structural unit found in a naturally occurring repeat protein adjacent to a repeat unit. Examples of capping sequences are described in International Patent Publication Nos. WO 2002/020565 and WO 2012/069655, in U.S. Patent Publication No. US 2013/0296221, and by Interlandi at al., J Mol Biol. 2008 Jan. 18; 375(3):837-54. Examples of N-terminal ankyrin capping modules (i.e., N-terminal capping repeats) include SEQ ID NOs: 69 to 72 and examples of ankyrin C-terminal capping modules (i.e., C-terminal capping repeats) include SEQ ID NO: 73 to 76.

### Nucleic Acids & Methods

In another aspect, the invention relates to a nucleic acid encoding the amino acid sequence of a recombinant binding protein of the present invention. In one aspect, the Invention relates to a nucleic acid encoding the amino acid sequence of a recombinant protein of the present invention. Furthermore, the invention relates to vectors comprising any nucleic acid of the Invention. Nucleic acids are well known to the skilled person in the art. In the examples, nucleic acids were used to produce designed ankyrin repeat domains or recombinant binding proteins of the invention in E. coli.

### Compositions, Uses and Methods of Treatment

In one aspect, the invention relates to a pharmaceutical composition comprising a recombinant binding protein and/or a designed ankyrin repeat domain of the present invention, and/or a nucleic acid encoding a recombinant binding protein and/or a designed ankyrin repeat domain of the present Invention, and optionally a pharmaceutically acceptable carrier and/or diluent.

In one aspect, the invention relates to a pharmaceutical composition comprising a recombinant binding protein or a nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below.

A pharmaceutical composition comprises a recombinant binding protein, and/or a designed ankyrin repeat domain, and/or a nucleic acid, preferably a recombinant binding protein and/or a nucleic acid, as described herein and a pharmaceutically acceptable carrier, excipient, or stabilizer, for example as described in Remington's Pharmaceutical Sciences 16^{th} edition. Osol, A. Ed., 1980.

Suitable carriers, diluents, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers, and preservatives. Other suitable carriers include any carrier that does not itself induce the production of antibodies harmful to the individual receiving the composition such as proteins, polysaccharides, polylactic acids, polyglycolic acids, polymeric amino acids, and amino acid copolymers. A pharmaceutical composition may also be a combination formulation, comprising an additional active agent, such as an anti-cancer agent or an anti-angiogenic agent, or an additional bioactive compound. The compositions to be used for in vivo administration must be aseptic or sterile. This is readily accomplished by filtration through sterile filtration membranes.

In one aspect, a pharmaceutical composition comprises at least one recombinant binding protein as described herein and a detergent such as nonionic detergent, a buffer such as phosphate buffer, and a sugar such as sucrose. In one aspect, such a composition comprises recombinant binding proteins as described above and PBS.

In another aspect, the invention provides a method of tumor-localized activation of T cells in a mammal, including a human, the method comprising the step of administering to said mammal the recombinant protein of the invention, the nudeic acid of the invention or the pharmaceutical composition of the invention.

In another aspect, the invention provides a method of treating a medical condition, the method comprising the step of administering to a patient in need thereof a therapeutically effective amount of the recombinant binding protein of the invention, the nucleic acid of the invention or the pharmaceutical composition of the invention.

In another aspect, the Invention provides a method of treating a medical condition, the method comprising the step of administering to a patient in need thereof a therapeutically effective amount of the Inventive recombinant binding protein further comprising a binding agent with binding specificity for a disease-associated antigen, a nucleic acid encoding said recombinant binding protein or a pharmaceutical composition comprising said binding protein.

In one aspect, the invention relates to a pharmaceutical composition, a recombinant binding protein, or a nucleic acid according to the present invention for use in the treatment of a disease. For that purpose, the pharmaceutical composition, the nucleic acid or the recombinant binding protein according to the present invention is administered to a patient In need thereof. In a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension, or emulsion, in association with the pharmaceutically acceptable excipients as defined above. The dosage and mode of administration will depend on the individual to be treated and the disease.

Further, any of the above-mentioned pharmaceutical composition, nucleic acid or recombinant protein is considered for use in the treatment of a disorder.

In one aspect, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one aspect, the invention relates to the use of the recombinant binding protein of the invention, the nucleic acid of the invention or the pharmaceutical composition of the invention, as medicament for the treatment of a disease. In one aspect, the invention relates to the use of the recombinant binding protein of the Invention, the nucleic acid of the Invention or the pharmaceutical composition of the invention for manufacturing of a medicament. In one aspect, the invention relates to the use of the recombinant binding protein of the invention, the nucleic acid of the Invention or the pharmaceutical composition of the Invention, for manufacturing of a medicament for the treatment of a disease. In one aspect, the invention relates to a process for the manufacturing of a medicament for the treatment of a disease, wherein the recombinant binding protein of the invention, the nucleic acid of the invention or the pharmaceutical composition of the invention is an active Ingredient of the medicament. In one aspect, the invention relates to a method of treatment of a disease using the recombinant binding protein of the invention, the nucleic acid of the invention or the pharmaceutical composition of the invention.

In one aspect the invention further provides a use of such a recombinant binding protein for treating a medical condition of a subject in need thereof.

As used herein, said medical condition or disease is a cancer, preferably a liquid tumor, more preferably leukemia, even more preferably acute myeloid leukemia (AML).

The recombinant binding protein of the present invention, nucleic acid of the invention or a pharmaceutical composition of the invention can also be used in combination with one or more other therapies known in the art. The term “use in combination with”, as used herein, shall refer to a co-administration, which is carried out under a given regimen. This includes synchronous administration of the different compounds as well as time-shifted administration of the different compounds (e.g., compound A is given once and compound B is given several times thereafter, or vice versa, or both compounds are given synchronously and one of the two is also given at later stages).

In one aspect, the invention relates to a kit comprising the recombinant binding protein of the invention.

In one aspect, the invention relates to a kit comprising a nucleic acid encoding the recombinant binding protein of the invention. In one aspect, the invention relates to a kit comprising the pharmaceutical composition of the invention. In one aspect, the invention relates to a kit comprising the recombinant protein of the invention, and/or the nucleic acid of the invention, and/or the pharmaceutical composition of the invention. In one aspect, the invention relates to a kit comprising the recombinant protein comprising an ankyrin repeat domain with binding specificity for CD33 of the invention, for example SEQ ID NOs: 1 to 16 and 77 to 78, and/or a nucleic acid encoding the recombinant protein comprising an ankyrin repeat domain with binding specificity for CD33, for example SEQ ID NOs: 1 to 16 and 77 to 78, and/or a pharmaceutical composition comprising the recombinant protein comprising an ankyrin repeat domain with binding specificity for CD33, for example SEQ ID NOs: 1 to 16 and 77 to 78. In one aspect, the invention relates to a kit comprising the recombinant protein comprising any one of the amino acid sequences of SEQ ID NOs: 1 to 59 and 77 to 81, and/or a nucleic acid encoding said recombinant protein, and/or a pharmaceutical composition comprising the recombinant protein.

In one aspect, the invention relates to a method for producing a recombinant protein of the present Invention. In one aspect, the invention relates to a method for producing a recombinant binding protein, for example a recombinant protein comprising the amino acid sequence of any one of SEQ ID NOs: 1 to 59 and 77 to 84, the method comprising the steps of (I) expressing said recombinant binding protein in a suitable host cell (e.g., bacteria), and (ii) purifying said recombinant binding protein (e.g., using chromatography). Said method may comprise additional steps. Such a method of producing a recombinant binding protein of the present invention is described in Example 1.

The invention is not restricted to the particular aspects described in the Examples. This specification refers to a number of amino acid sequences, nucleic acid sequences and SEQ ID NOs that are disclosed in the appended Sequence Listing, which is herewith incorporated by reference In its entirety.

### Definitions

Unless defined otherwise herein, all technical and scientific terms used herein shall have the meanings that are commonly understood by those of ordinary skill in the art to which the present invention belongs.

Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, aspects also are contemplated “consisting of” or “consisting essentially of” the feature. The use of any and all examples, or exemplary language (e.g., “such as”) provided herein, Is Intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g., by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled In the art. For example, a recombinant DNA molecule (e.g., produced by gene synthesis) encoding a polypeptide can be cloned into a bacterial expression plasmid (e.g., pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g., Escherichia coli), these bacteria can produce the polypeptide(s) encoded by this recombinant DNA. The correspondingly produced polypeptide or protein Is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise an ankyrin repeat domain with binding specificity for CD33.

Furthermore, any such binding protein may comprise additional polypeptides (such as e.g., polypeptide tags, peptide linkers, fusion to other proteinaceous domains with binding specificity, cytokines, hormones, or antagonists), or chemical modifications (such as coupling to polyethylene-glycol, toxins (e.g., DM1 from immunogen), small molecules, antibiotics and alike) well known to the person skilled in the art. A binding protein of the instant invention may comprise a localizer molecule.

The term “binding domain” means a protein domain exhibiting binding specificity for a target. Preferably, said binding domain is a recombinant binding domain.

As used herein, the term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, the target is CD33. More preferably, the target is human CD33.

In the context of the present invention, the term “polypeptide” relates to a molecule consisting of a chain of multiple. i.e., two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

Patent application WO2002/020565 and Forrer at al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO20021020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “ankyrin repeat domain” refers to a repeat domain comprising two or more consecutive ankyrin repeat modules as structural units. Ankyrin repeat domains may be modularly assembled into larger ankyrin repeat proteins, optionally with half-life extension domains, using standard recombinant DNA technologies (see, e.g., Forrer, P., at al., FEBS letters 539, 2-6, 2003. WO2002/020565, WO2016/156596, WO2018/054971).

The term “designed” as used In designed repeat protein, designed repeat domain and the like refers to the property that such repeat proteins and repeat domains, respectively, are man-made and do not occur in nature. The binding proteins of the instant invention are designed repeat proteins and they comprise at least one designed ankyrin repeat domain. Preferably, the designed repeat domain is a designed ankyrin repeat domain.

The term “target Interaction residues” refers to amino acid residues of a repeat module, which contribute to the direct interaction with a target.

The term “framework residues” refers to amino acid residues of a repeat module, which contribute to the folding topology, i.e., which contribute to the fold of said repeat module or which contribute to the interaction with a neighboring module. Such contribution may be the interaction with other residues in the repeat module, or the Influence on the polypeptide backbone conformation as found in α-helices or β-sheets, or the participation in amino acid stretches forming linear polypeptides or loops. Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g., the family of ankyrin repeat proteins.

Furthermore, each repeat module comprised in a repeat domain may comprise a “repeat sequence motif” deduced from homologous repeat modules obtained from repeat domains selected on a target, e.g., as described in Example 1, and having the same target specificity.

Accordingly, the term “ankyrin repeat module” refers to a repeat module, which is originally derived from the repeat units of naturally occurring ankyrin repeat proteins. Ankyrin repeat proteins are well known to the person sidled in the art. Designed ankyrin repeat proteins have been described previously; see, e.g., International Patent Publication Nos. WO2002/020585, WO2010/060748, WO2011/135067, WO2012/069654, WO2012/069655, WO2014/001442, WO2014/191574, WO2014/083208, WO2016/156596, and WO2018/054971, all of which are Incorporated by reference in their entireties. Typically, an ankyrin repeat module comprises about 31 to 33 amino acid residues that form two alpha helices, separated by loops.

Repeat modules may comprise positions with amino acid residues which have not been randomized In a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline.

The term “repeat sequence motif” refers to an amino acid sequence, which is deduced from one or more repeat modules. Preferably, said repeat modules are from repeat domains having binding specificity for the same target Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “has binding specificity for a target”, “specifically binding to a target”, “binding to a target with high specificity”, “specific for a target”. “target specificity”, or “specifically binds” and the like means that a binding protein or binding domain binds in PBS to a target with a lower dissociation constant (i.e., it binds with higher affinity) than it binds to an unrelated protein such as the E. coli maltose binding protein (MBP). Preferably, the dissociation constant (“K_{D}”) in PBS for the target is at least 10²; more preferably, at least 10³; more preferably, at least 10⁴; or more preferably, at least 10⁵ times lower than the corresponding dissociation constant for MBP. Methods to determine dissociation constants of protein-protein Interactions, such as surface plasmon resonance (SPR) based technologies (e.g., SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS. A typical and preferred determination of dissociation constants (K_{D}) of the inventive recombinant binding proteins with binding specificity for CD33 by Surface Plasmon Resonance (SPR) analysis is described in Example 2. A variety of assay formats may be used to select or characterize a binding moiety that specifically binds a drug molecule of Interest. For example, solid-phase ELISA immunoassay, immunoprecipitation, BIAcore™ (GE Healthcare, Piscataway, NJ), fluorescence-activated cell sorting (FACS), Octet™ (ForteBio, Inc., Menlo Park, CA) and Western blot analysis are among many assays that may be used to identify a binding moiety that specifically binds to a target drug molecule. Typically, a specific or selective binding will be at least twice the background signal or noise and more typically more than 10 times the background signal. More particularly, a binding agent is said to “specifically bind” a target when the equilibrium dissociation constant (K_{D}) value is <1 μM, such as <500 nM, <100 nM, <10 nM, <1 nM, <100 pM or <10 pM.

The term “binding agent” or “binding moiety” refers to any molecule capable of specifically binding a target molecule. Binding agents include, for example, antibodies, antibody fragments, aptamers, peptides (e.g., Williams et al., J Biol Chem 266:5182-5190 (1991)), alternative scaffolds, antibody mimics, repeat proteins, e.g., designed ankyrin repeat proteins, receptor proteins and any other naturally occurring interaction partners of the target molecule, and can comprise natural proteins and proteins modified or genetically engineered, e.g., to include non-natural residues and/or to lack natural residues.

The term “PBS” means a phosphate buffered water solution containing 137 mM NaCl, 10 mM phosphate and 2.7 mM KCl and having a pH of 7.4.

Preferably, clearance, and/or exposure, and/or terminal half-life are assessed in a mammal, more preferably mouse and/or cynomolgus monkey, more preferably cynomolgus monkey. Preferably, when measuring the clearance, and/or exposure, and/or terminal half-life in mouse, the evaluation is done considering the data up to 48 h post-injection. More preferably, the evaluation of terminal half-life In mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is well known to the person skilled in the art Preferably, pharmacokinetic companion is done at any dose, more preferably at equivalent dose (i.e., same mg/kg dose) or equimolar dose (i.e., same mol/kg dose), more preferably at equimolar dose (i.e., same mol/kg dose). It is understood by the person skilled in the art that equivalent and/or equimolar dosing in animals is subject to experimental dose variations of at least about 20%, more preferably about 30%, about 40%, about 50%, about 60%, about 70%, about 80%, about 90%, or about 100%.

Preferably, a dose used for pharmacokinetic measurement is selected from about 0.001 to about 1000 mg/kg, more preferably about 0.01 to about 100 mg/kg, more preferably about 0.1 to about 50 mg/kg, more preferably about 0.5 to about 10 mg/kg.

The term “CD3” or “Cluster of Differentiation 3” refers to a multimeric protein complex composed of four distinct polypeptide chains, epsilon (ε), gamma (γ) and zeta (ζ) that assemble as three pairs (εγ, εδ, ζζ). The CD3 complex serves as a T cell co-receptor that associates non-covalently with the T cell receptor. It may refer to any form of CD3, as well as to variants, isoforms, and species homologs thereof that retain at least a part of the activity of CD3. Accordingly, a binding protein, as defined and disclosed herein, may also bind CD3 from species other than human. In other cases, a binding protein may be completely specific for the human CD3 and may not exhibit species or other types of cross-reactivity. Unless indicated differently, such as by specific reference to human CD3, CD3 includes all mammalian species of native sequence CD3, e.g., human, canine, feline, equine and bovine. The amino acid sequences of human CD3 gamma, delta and zeta chains are shown in NCBI (www.ncbi.nlm.nih.gov/) Ref. Seq. NP_000064.1, NP_000723.1 and NP_932170.1 respectively.

The term “CD3-expressing cells” as used herein refers to any cells expressing CD3 (cluster of differentiation 3) on the cell surface, including, but not limited, to T cells such as cytotoxic T cells (CD8+ T cells) and T helper cells (CD4+ T cells).

The term “CD33” refers to myeloid cell surface antigen CD33, which is a sialic-acid-binding immunoglobulin-like lectin (Siglec) that plays a role in mediating cell-cell interactions and in maintaining immune cells in a resting state. The amino acid sequence of human CD33 (hCD33) is shown in UniProt (www.unirot.org) Ref. No. P20138.

The term “tumor-localized activation of T cells” means that T calls are activated preferentially in tumor tissue as compared to a non-tumor tissue.

Furthermore, the term “peptide” also encompasses peptides modified by, e.g., glycosylation, and proteins comprising two or more polypeptide chains, each of length of 4 to 600 amino acids long, cross-linked by, e.g., disulfide bonds, such as, e.g., Insulin and immunoglobulins. The term “chemical or biochemical agent” is intended to include any naturally occurring or synthetic compound that may be administered to a recipient. In a preferred aspect, the localizer is a target-specific ankyrin repeat domain.

The term “medical condition” (or disorder or disease) includes autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder such as a neoplastic disease. A “medical condition” may be one that is characterized by inappropriate cell proliferation. A medical condition may be a hyperproliferative condition. The invention particularly relates to a method of treating a medical condition, the method comprising the step of administering, to a patient in need of such treatment, a therapeutically effective amount of a recombinant binding protein or said pharmaceutical composition of the invention. In a preferred aspect said medical condition is a neoplastic disease. The term “neoplastic disease”, as used herein, refers to an abnormal state or condition of cells or tissue characterized by rapidly proliferating cell growth or neoplasm. In one aspect said medical condition is a malignant neoplastic disease. In one aspect said medical condition is a cancer, preferably leukemia, more preferably acute myeloid leukemia. The term “therapeutically effective amount” means an amount that is sufficient to produce a desired effect on a patient.

The term “antibody” means not only Intact antibody molecules, but also any fragments and variants of antibody molecules that retain immunogen-binding ability. Such fragments and variants are also well known in the art and are regularly employed both in vitro and in vivo. Accordingly, the term “antibody” encompasses intact immunoglobulin molecules, antibody fragments such as, e.g., Fab, Fab′, F(ab′)₂, and single chain V region fragments (scFv), bispecific antibodies, chimeric antibodies, antibody fusion polypeptides, and unconventional antibodies.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CMIL), small lymphocytic lymphoma (SLL), malignant mesothelioma, colorectal cancer, or gastric cancer.

### EXAMPLES

Starting materials and reagents disclosed below are known to those skilled in the art, are commercially available and/or can be prepared using well-known techniques.

Materials Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g., E. coli XL1-blue (Stratagene, USA) or BL21 (Novagen, USA).

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g., in U.S. Pat. No. 7,417,130; Binz et al., J. Mol. Biol. 332, 489-503, 2003; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 69, 70, or 71) or a randomized N-terminal capping module (e.g. according to SEQ ID NO: 72), and a fixed C-terminal capping module (e.g. the C-terminal capping module of SEQ ID NO: 73, 74, or 75) or a randomized C-terminal capping module (e.g. according to SEQ ID NO: 76). Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g., 1 to 20 amino acids) inserted in one or more beta-turns of an ankyrin repeat domain. Any such N-terminal capping module of an ankyrin repeat protein library preferably possesses the RILLAA, RILLKA or RELLKA motif (e.g., present from position 19 to 24 in SEQ ID NO: 1) and any such C-terminal capping module of an ankyrin repeat protein library preferably possesses the KLN, KLA or KAA motif (e.g., present at the last three amino acids in SEQ ID NO: 1). SEQ ID NO: 69 to 71 provide examples of N-terminal capping modules comprising the RILLAA, RILLKA or RELLKA motif, and of SEQ ID NO: 73 to 75 provide examples of C-terminal capping modules comprising the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TW-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

### Example 1: Selection of Binding Proteins Comprising an Ankyrin Repeat Domain with Binding Specificity for CD33

Using ribosome display (Hanes, J. and Plückthun, A., PNAS 94, 4937-42, 1997), many ankyrin repeat proteins with binding specificity for human CD33 (hCD33) (UniProt ID P20138) were selected from DARPin® libraries similar as described by Binz et al. 2004 (loc. cit.). The binding of the selected clones to recombinant human CD33 targets (full-length extracellular domain (ECD) of CD33 and a splice variant of the ECD of CD33) was assessed by crude extract Homogeneous Time Resolved Fluorescence (HTRF), indicating that hundreds of hCD33-specific binding proteins were successfully selected. For example, the ankyrin repeat domains of SEQ ID NOs: 1, 3, 5, 7-9, and 14-16 constitute amino acid sequences of selected binding proteins comprising an ankyrin repeat domain with binding specificity for hCD33.

### Selection of CD33-Specific Ankyrin Repeat Proteins by Ribosome Display

The selection of hCD33-specific ankyrin repeat proteins was performed by ribosome display (Hanes and Plückthun, loc. cit.) using biotinylated extracellular domain (ECD) of human CD33 as target protein, libraries of ankyrin repeat proteins as described above, and established protocols (See, e.g., Zahnd, C., Amstutz, P. and Plückthun, A., Nat Methods 4, 69-79, 2007). CD33 targets (Evitria) contained a C-terminal Fc Tag and an Avi tag and were biotinylated using the enzyme BirA-GST. Two different forms of CD33 were used for selection: Full-length ECD of CD33 (residues 18-259) (SEQ ID NO: 60) containing both variable and constant domains of CD33, and a splice variant of the ECD of CD33 (residues 140-259) (SEQ ID NO: 61) containing only the constant domain of CD33. In total four rounds of standard ribosome selections were employed, using decreasing target concentration and increasing washing stringency to increase selection pressure from round 1 to round 4 (Binz at al. 2004, loc. cit.).

A deselection strategy was applied in each round by using Streptavidin and Neutravidin Beads In conjunction with biotinylated non-CD33 Fc domain. The number of reverse transcription (RT)-PCR cycles after each selection round was constantly reduced from 45 to 28 adjusting to the yield due to enrichment of binders.

To enrich high affinity CD33-specific ankyrin repeat proteins, the output from the fourth round of standard ribosome display selection (above) was subjected to an off-rate selection round with increased selection stringency (Zahnd, 2007, loc. cit.). A final standard selection round was performed after the off-rate selection round to amplify and recover the off-rate selected binding proteins. In these last two selection rounds, the number of RT-PCR cycles was 30 and 35, respectively.

In total three different selection approaches have been conducted as described above with the following differences: In the first approach, selections were performed only against the ECD of the full-length CD33 protein. In a second approach, targets of full-length CD33 ECD and splice variant ECD were alternated in each round. In the third approach, a competitive elution step was applied using the condition of the first approach by adding HIM-3-4 CD33 binding antibody (BD Pharmingen™). From each approach, binders against full-length CD33 and/or its splice variant were generated.

### Selected Clones Bind Specifically to Human CD33 as Shown by Crude Extract HTRF

Individual selected ankyrin repeat proteins specifically binding to hCD33 in solution were identified by a Homogeneous Time Resolved Fluorescence (HTRF) assay using crude extracts of ankyrin repeat protein-expressing Escherichia coli cells using standard protocols. Ankyrin repeat protein clones selected by ribosome display were cloned into derivatives of the pQE30 (Qiagen) expression vector (pMPDV045), which contains a C-terminal CD3-specific ankyrin repeat domain followed by a Flag tag, transformed into E. coli XL1-Blue (Stratagene), plated on LB-agar (containing 1% glucose and 50 pa/ml ampicillin) and then incubated overnight at 37° C. Single colonies were picked into a 96 well plate (each clone in a single well) containing 160 μl growth medium (TB containing 1% glucose and 50 μg/ml ampicillin) and incubated overnight at 37° C., shaking at 800 rpm. 150 μl of fresh TB medium containing 50 pg/ml ampicillin was inoculated with 8.5 μl of the overnight culture in a fresh 96-well plate. After incubation for 120 minutes at 37° C. and 700 rpm, expression was induced with IPTG (0.5 mM final concentration) and continued for 4 hours. Cells were harvested and the pellets were frozen at −20° C. overnight before resuspension in 8 μl μl B-PERII (Thermo Scientific) and incubation for 1 hour at room temperature with shaking (900 rpm). Then, 160 μl PBS was added and cell debris was removed by centrifugation (3220 g for 15 min).

The extract of each lysed done was applied as a 1:1000 dilution (final concentration) in PBSTB (PBS supplemented with 0.1% Tween 200 and 0.2% (w/v) BSA, pH 7.4) together with 6 nM (final concentration) biotinylated hCD33 (full-length or splice variant of ECD CD33), 1:400 (final concentration) of anti-strep-Tb HTRF antibody—FRET donor conjugate (Cisbio) and 1:400 (final concentration) of anti-6His-D2 antibody FRET acceptor conjugate (Cisbio) to a well of 384 well plate and incubated for 60 minutes at RT. The HTRF was read-out on a Tecan M1000 using a 340 nm excitation wavelength and a 665±10 nm emission filler. Screening of several hundred clones by such a crude cell extract HTRF revealed ankyrin repeat domains with specificity for hCD33. Amino acid sequences of selected ankyrin repeat domains that specifically bind to hCD33 are provided in SEQ ID NOs: 1, 3, 5, 7-9, and 14-16.

TABLE 1a

DARPin ® protein #1 (SEQ ID NO: 1)
DARPin ® protein #3 (SEQ ID NO: 3)
DARPin ® protein #5 (SEQ ID NO: 5)
DARPin ® protein #7 (SEQ ID NO: 7)
DARPin ® protein #8 (SEQ ID NO: 8)
DARPin ® protein #9 (SEQ ID NO: 9)
DARPin ® protein #14 (SEQ ID NO: 14)
DARPin ® protein #15 (SEQ ID NO: 15)
DARPin ® protein #16 (SEQ ID NO: 16)

These DARPin® proteins optionally comprise additionally a G, an S or a GS sequence at their N-terminus.

Engineering of Additional Ankyrin Repeat Proteins with Binding Specificity for hCD33

TABLE 1b

DARPin ® protein #2 (SEQ ID NO: 2)
DARPin ® protein #4 (SEQ ID NO: 4)
DARPin ® protein #6 (SEQ ID NO: 6)
DARPin ® protein #10 (SEQ ID NO: 10)
DARPin ® protein #11 (SEQ ID NO: 11)
DARPin ® protein #12 (SEQ ID NO: 12)
DARPin ® protein #13 (SEQ ID NO: 13)

SEQ ID NOs: 2, 4, 6 and 10-13 are ankyrin repeat proteins with binding specificity for hCD33 that were engineered based on the sequence of DARPin® protein #1 (SEQ ID NO: 1), DARPin® protein #3 (SEQ ID NO: 3), DARPin® protein #5 (SEQ ID NO: 5) and DARPin® protein #9 (SEQ ID NO: 9), respectively.

For SEQ ID NO: 1 and SEQ ID NO: 3, the sequence was modified in order to reduce the number of aromatic residues and change the surface charges. In both N-terminal capping modules, the RILLAA motiv was replaced by RILLKA and Aspartate (position 18) was replaced by Leucine. In both C-terminal capping modules, Glutamate (position18) was replaced by Glutamine. For SEQ ID NO: 3, an additional Phenylalanine (position 14) was replaced by a Valine in the N-terminal capping module. For SEQ ID NO:1, an additional Tryptophane (position 7) was replaced by a Valine in the N-terminal capping module, and the EDIA motiv in the second internal repeat (position 18-21) was replaced by LEIV.

Engineered variants did not alter T-cell killing (assessed in combination with other TAA- and CD3-binding ankyrin repeat domains) compared to parental version by more than factor two measured In a standard LDH killing assay after 48 h of incubation using PanT and MOLM-13 cells at a ratio of 5:1.

For SEQ ID NO: 9, the sequence was modified in order to reduce the number of aromatic residues, change the surface charges and/or optimize the framework. In all N-terminal capping modules, the RILLAA motiv was replaced by RELLKA. In three variants, either Valine (position12) in the second internal repeat was removed in order to get a standard ankyrin repeat protein framework as described by Binz et al., 2004, Nature Biotechnology, or Glycine (position 18) in the C-terminal capping repeat was replaced by Glutamate, or a combination of both was introduced. In one variant, Tyrosine (position 7) In the N-terminal capping module was replaced by Valine. Engineered variants maintained binding against hCD33 as measured by HTRF and cell binding against MOLM13 cells as measured by FACS.

For SEQ ID NO: 5, Serine (position 6) was replaced by a Glycine in the N-terminal capping module. Engineered variants did not alter T-cell killing (assessed in combination with a CD3 binding ankyrin repeat domain) compared to parental version by more than factor two measured in a standard LDH killing assay after 48 h of incubation using PanT and MOLM-13 cells at a ratio of 5:1.

### Expression of CD33-Specific Ankyrin Repeat Proteins

For further analysis, the selected clones showing specific human CD33 binding in the crude cell extract HTRF, as described above, were expressed in E. coli cells, with a His-Tag (SEQ ID NO: 85) fused to their N-terminus for easy purification. Expressed proteins were purified according to standard protocols. 0.11 ml of stationary overnight cultures (TB, 1% glucose, 50 mg/n of ampicillin; 37° C.) were used to inoculate 0.99 ml cultures in 96-deep-well plate (TB, 50 mg/l ampicillin, 37° C.). After 2 hours incubation at 37° C. (700 rpm), the cultures were Induced with 0.5 mM IPTG and incubated at 37° C. for 6 h with shaking (900 rpm). Cells were harvested and the pellets were frozen at −20° C. overnight before resuspensions in 50 μl B-PERII (Thermo Scientific) supplemented with DNAse I (200 Units/ml) and Lysozyme (0.4 mg/ml) and incubation for one hour at room temperature with shaking (900 rpm). Then, 60 μl low salt sodium phosphate buffer was added and cell debris was removed by centrifugation (3220 g for 15 min). In total, eight individual expressions were pooled, before removal of cell debris by centrifugation (3′200 g for 60 min at 4° C.). Supernatant was filtered using a MultiScreen filter plate (Millipore) before purification using a 96-well Thermo HisPur cobalt spin plates and rebuffering the proteins solution using 98-well Thermo Zeba spin desalting plate in PBS. Purified proteins were soluble and monomeric In PBS using a standard Sephadex 150/5 column on an Agilent 1200 HPLC system.

### Generation of Affinity Matured CD33-Specific Binding Proteins

In further development of the Initially Identified CD33 specific binding proteins, new variants with very high affinity to and/or very low off-rate from target protein were generated using affinity maturation. Thereby, one initially identified CD33 specific binding protein DARPin® protein #2 (the “parental” binding protein) was selected as a suitable starting point for affinity maturation. The affinity maturation procedure entailed saturation mutagenesis of each randomized position of the ankyrin repeat domain used as a starting point. Sequences generated by the affinity maturation procedure were screened for lower off-rates by competition HTRF. In short: Single amino acid point mutations variants were generated by standard QuikChange PCR on parental plasmid (pMCHE1190) using primers with a single NNK degenerated codon to introduce all 20 amino acids at a potential binding position. Crude extracts (CE) of ankyrin repeat proteins, containing an N-terminal His-tag (SEQ ID NO: 85) were incubated with the biotinylated target before addition of excess of non-lag-tagged parental CD33-specific binding proteins and measurement of HTRF signal over time. Beneficial mutations, identified based on higher HTRF signals compared to parental clone, were combined in the binding proteins by protein engineering. This way, affinity matured protein #29 and DARPin® protein #30 were generated, originating from DARPin® protein #2.

Such affinity matured CD33-specific binding domains were then subcloned into a derivative of the pQE30 (Qiagen) expression vector, resulting in expression constructs encoding an N-terminal His-tag (SEQ ID NO:85), followed by the CD33 specific binding domain of SEQ ID NO: 77 or 78, a peptide linker (SEQ ID NO:65) and a C-terminal CD3 binding domain of SEQ ID NO:57. These constructs in T-cel engager format were expressed in E. coli cells and purified using their His-tag according to standard protocols. Proteins were tested for dose-dependent in vitro T-cell activation and tumor cell king in assays using primary T-cells isolated from healthy donor PBMCs as effector cells (E) and Molm-13-N1 tumor cells as target cells (T) (E:T ratio of 5:1). Assay incubation of co-culture was for 48 h and analysis by Flow Cytometry and LDH release. All constructs were expressed in E. coli cells and purified using their His-tag according to standard protocols.

### Example 2: Determination of Dissociation Constants (K_{D}) of Recombinant Ankyrin Repeat Proteins with Binding Specificity for Human CD33 by Surface Plasmon Resonance (SPR) Analysis

The binding affinities of purified ankyrin repeat proteins on recombinant human CD33 target were analyzed using a ProteOn instrument (BioRad) and the measurement was performed according standard procedures known to the person skilled in the art. For that, DARPin® proteins #1, 3, 5, 7-9, and 14-16 of the invention were subcloned and expressed as described above in derivatives of the pQE30 (Qiagen) expression vector, resulting in constructs containing an N-terminal His-tag and a CD33-specific ankyrin repeat domain, followed by one of the five CD3-specific ankyrin repeat domains listed in the Sequence Listing.

Briefly, SPR measurements were performed using a ProteOn XPR36 instrument (BioRad). The running buffer was PBS pH 7.4 containing 0.005% Tween 20® (PBST). The bio.hCD33 full-length target (SEQ ID NO: 60) was immobilized on NLC chips (BioRad) to a level of 540 RU and the spliced variant (SEQ ID NO: 61) to a level of 560RU. The interaction of the 96 well purified CD33-specific binding proteins in TCE format to the full-length and the spliced variant of CD33 ECD were measured by injecting the binding protein at 50 nM with an association of 120s and dissociation of 1200s using a constant flow of 100 U/min. The target was regenerated between the individual measurements using 2M MgCl₂. The signals were double referenced against the running buffer (PBST) treated control lane.

Table 2a shows K_{D} values obtained for DARPin® proteins #1, 3, 5, 7-9, and 14-16 in TCE format (i.e., formatted with a CD3-specific ankyrin repeat domain). Dissociation constants (K_{D}) were calculated from the estimated on- and off-rates using standard procedures known to the person skilled in the art. K_{D} values of the binding interactions of selected ankyrin repeat proteins with human truncated and full-length CD33 were determined by single trace SPR to be in the range of 0.47-17 nM (see Table 2a). The values in Table 2a are averages of multiple replicates.

TABLE 2a
K_{D} values of ankyrin repeat protein - human CD33 interactions

| | SPR against splice | SPR against full- |
|---|---|---|
| DARPin ® protein | variant CD33 | length CD33 |
| (in TCE format) | K_{D} | K_{D} |
| DARPin ® protein # 7 | no binding | 4.7E−10 |
| DARPin ® protein # 9 | 1.9E−09 | 7.6E−10 |
| DARPin ® protein # 15 | no binding | 1.70E−08 |
| DARPin ® protein # 3 | no binding | 7.4E−09 |
| DARPin ® protein # 8 | no binding | 2.3E−09 |
| DARPin ® protein # 1 | no binding | 4.8E−09 |
| DARPin ® protein # 5 | no binding | 1.2E−08 |
| DARPin ® protein # 16 | no binding | no binding |
| DARPin ® protein # 14 | no binding | 6.4E−10 |

Moreover, the binding affinities of two additional purified ankyrin repeat proteins on recombinant human CD33 target were measured and analyzed with a similar procedure as described above for DARPin proteins #1, #3, #6, #7-#9 and #14-#16. Briefly, bio.hCD33 was coaled on NLC chip (BioRad) to a level of 1180 RU. DARPin® protein #29 and DARPin® protein #30 were applied as analyte at a single concentration of 100 nM with a 120 s association and 1200 s dissociation phase at a constant flow of 100 ul/min. The target was regenerated with 4M MgCl2. The signals were double referenced against the running buffer (PBST-PBS pH 7.4 containing 0.005% Tween 200) treated control lane of L1 and A6. The 1:1 Langmuir model was used for the fitting. The K_{D} values obtained in this study are summarized in Table 2b.

Table 2b shows K_{D} values of CD33-specific ankyrin repeat proteins of the invention binding to bio.hCD33 full length target. K_{D} values were calculated from the estimated on- and off-rates using standard procedures known to the person skilled in the art. The values In Table 2b are averages of multiple replicates.

| Protein | K_{D} [M] |
|---|---|
| DARPin protein #29 | 6.2E−10 |
| DARPin protein #30 | 8.8E−10 |

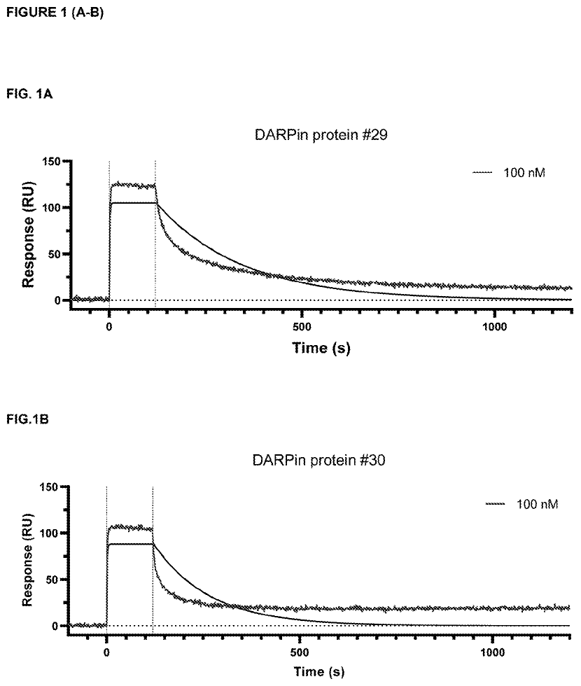

FIG. 1 (A-B) shows Surface Plasmon Resonance (SPR) analysis of ankyrin repeat proteins binding to human CD33: FIG. 1A SPR analysis for DARPin® protein #29 and FIG. 1B SPR analysis for DARPin® protein #30.

### Example 3: Pharmacokinetic Analysis of CD33-Specific Ankyrin Repeat Proteins in Female BALB/c Mice

In order to determine whether a CD33-specific ankyrin repeat domain of the invention can have an appropriate serum half-life In vivo for it to be useful for the development of therapeutic agents, the pharmacokinetic profiles of DARPin® protein #2, DARPin® protein #29 and DARPin® protein #30 are analyzed in mice. For that, DARPin constructs are subcloned and expressed as described above into derivatives of the pOE30 (Qiagen) expression vector, encoding an N-terminal His-tag (SEQ ID NO:85), an HSA binding ankyrin repeat domain (SEQ ID NO: 53) for half-life extension, a peptide linker (such as SEQ ID NO:65), and at the C-terminal one of the CD33-specific binding domains. For example, expression vectors encoding the following ankyrin repeat proteins are constructed:

### In Vivo Administration and Sample Collection

DARPin® protein #2, DARPin® protein #29 and DARPIn® protein #30, formatted with a human serum albumin specific ankyrin repeat domain (SEQ ID NO: 53) as described above, are administered as a single intravenous bolus injection Into the tail vein of 6 mice for each ankyrin repeat fusion protein. The target dose level is 1 mg/kg with an application volume of 5 mL/kg. Ankyrin repeat fusion proteins are formulated in phosphate-buffered saline (PBS) solution.

Mice are split Into 2 groups with equal numbers of animals. Four serum samples are collected from each mouse. Blood samples for pharmacokinetic investigations are collected from the saphenous vein at 5 min, 4 h, 24 h, 48 h, 76 h, 96 h and 168 h post compound administration. Blood Is kept at room temperature to allow clotting followed by centrifugation and collection of serum.

### Bioanalytics by ELISA to Measure Ankyrin Repeat Proteins in Serum Samples

One hundred pi per well of 10 nM polyclonal goat anti-rabbit IgG antibody (Ab18) in PBS is coated onto a NUNC Maxisorb ELISA plate overnight at 4° C. After washing with 300 μl PBST (PBS supplemented with 0.1% Tween20) per well five times, the wells are blocked with 300 μl PBST supplemented with 0.25% Casein (PBST-C) for 1 h at room temperature (RT) on a Heidolph Titramax 1000 shaker (450 rpm). Plates are washed as described above. 100 μl 5 mol/L rabbit anti-DARPin® 1-1-1 antibody In PBST-C is added and the plates are incubated at RT (22° C.) with orbital shaking (450 rpm) for 1 h. Plates are washed as described above.

One hundred μl of diluted serum samples (1:20-1:312500 in 1:5 dilution steps) or ankyrin repeat protein standard curve samples (0 and 50-0.0008 nmol/L in 1:3 dilution steps) are applied for 2 h, at RT, shaking at 450 rpm. Plates are washed as described above.

Wells are then incubated with 100 μl murine anti-RGS-His-HRP IgG (Ab06, 1:2000 in PBST-C) and incubated for 1 h, at RT, 450 rpm. Plates are washed as described above. The ELISA is developed using 100 μl/well TMB substrate solution for 5 minutes and stops by the addition of 100 μl 1 mol/L H₂SO₄. The difference between the absorbance at 450 nm and the absorbance at 620 nm is calculated. Samples are measured in duplicate on two different plates.

### Pharmacokinetic Analysis

Pharmacokinetic data analysis Is performed at Molecular Partners using Version 7.0 of the WinNonlin program as part of Phoenix 64, Pharsight, North Carolina. Calculation of the pharmacokinetic parameters based on the mean concentration-time data of the animals dosed via intravenous bolus injection is performed with non-compartmental analysis (NCA model 200-202, IV bolus, linear trapezoidal linear interpolation). Pharmacokinetic parameters are calculated, such as the following: AUCinf, AUClast, AUC_% extrapol, Cmax, Tmax, CI_pred, Vss_pred, t1/2 Maximum serum concentrations (Cmax) and the times of their occurrence (Tmax) are obtained directly from the serum concentration-time profiles. The area under the serum concentration-time curve (AUCinf) is determined by the linear trapezoidal formula up to the last sampling point (Tlast) and extrapolation to infinity assuming mono-exponential decrease of the terminal phase. The extrapolation up to infinity is performed using Clast/λz, where λz denotes the terminal rate constant estimated by log linear regression and Clast denotes the concentration estimated at Tlast by means of the terminal log-linear regression. Total serum clearance (CI_pred) and the apparent terminal half-life are calculated as follows: CI_pred=i.v. dose/AUCinf and t1/2=In2/λz. The steady-state volume of distribution Vss is determined by: Vss=i.v. dose·AUMCinf/(AUCinf)2. AUMCinf denotes the total area under the first moment of drug concentration-time curve extrapolated to infinity using the same extrapolation procedure as described for calculation of AUCinf. To calculate PK parameters based on concentrations given in nmol/L dose, values given as mg/kg are converted to nmol/kg by using the molecular weight of the ankyrin repeat proteins. Table 3 shows approximate predicted half-lives of three ankyrin repeat proteins of the invention, DARPin® protein #2, DARPin® protein #29 and DARPin® protein #30, formatted with a human serum albumin-specific ankyrin repeat domain as described above.

TABLE 3
Half-life (t½) of three exemplary
HSA × CD33-specific ankyrin repeat proteins

| | | DARPin ® | DARPin ® | DARPin ® |
|---|---|---|---|---|
| parameter | unit | protein #2 | protein #29 | protein #30 |
| HL_Lambda_z | h | ~23 | ~27 | ~33 |
| (half- life) | | | | |

In conclusion, CD33-specific ankyrin repeat domains of the invention can be combined with a half-life extending moiety, such as, e.g., a serum albumin-specific binding domain, to achieve an appropriate serum half-life in vivo for them to be useful for the development of therapeutic agents.

### Example 4: Determination of Binding of Ankyrin Repeat Proteins of the Invention to CD33-Expressing Tumor Cells

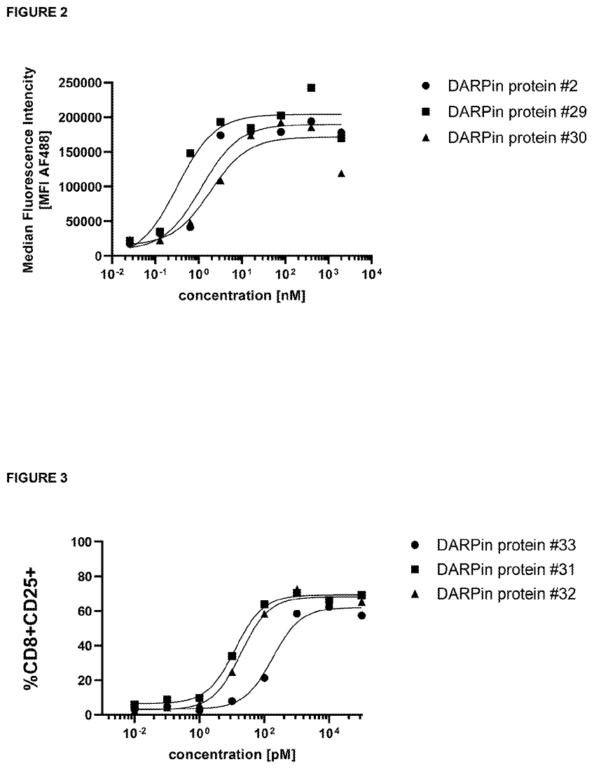

Binding of binding proteins of the invention to CD33 expressed on the surface of cells was analyzed by fluorescence activated cell sorting (FACS) flow cytometry. For this purpose. CD33-expressing tumor cells (Molm-13 N1) were seeded at 100,000 cells per well in a 96 well plate. DARPin® protein #2, DARPin protein #29, and DARPin® protein #30, in monospecific format, were titrated down In 1:5 dilution ratio starting at 2000 nM. Tumor cells were resuspended with diluted DARPin proteins and incubated for 60 minutes at 4° C. The assay was performed in PBS Including 2% fetal bovine serum without human serum albumin (HSA). After washing twice with Phosphate Buffer saline (PBS), DARPin® protein specific tumor call binding was detected by adding unlabeled primary anti-rabbit DARPin® antibody (anti-rabbit 1-1-1 antibody, CePower) at 2 μg/ml. An incubation step of at least 30 minutes at 4° C. followed. Afterwards, cells were washed with PBS and a secondary goat anti-rabbit antibody labelled with Alexa Fluor 488 antibody (ThermoFisher) at 2 ug/ml was added. The same incubation conditions applied. Finally, the cells were washed twice and resuspended in Cytofix fixation buffer (BD Biosciences) for 15 min at room temperature (RT). Median fluorescence intensities (MFI) of Alexa Fluor 488 DARPin® protein labelled tumor cells were measured by Attune NXT (ThermoFisher) using FlowJo software for analyses and GraphPad Prism 8 for data plotting (FIG. 2 shows binding curves of DARPin® protein #2, DARPin protein #29, and DARPin® protein 30 to CD33 expressing tumor cells). Table 4 shows a quantification of the binding of the three exemplary ankyrin repeat proteins to CD33 expressed on cells, as represented by their EC50 values.

TABLE 4

| Protein | EC50[nM] |
|---|---|
| DARPin protein #2 | 1.2 |
| DARPin protein #29 | 0.32 |
| DARPin protein #30 | 1.9 |

In conclusion. CD33-specific binding proteins of the invention bind to CD33 expressed on the surface of cells with an EC50 of about 2 nM or below.

### Example 5: Assessment of Specificity and Potency of CD33-Specific Ankyrin Repeat Proteins of the Invention with a Target-Specific Short-Term T Cell Activation Assay

Specificity and potency of previously described exemplary CD33-specific ankyrin repeat proteins of of the invention were assessed in an in vitro short-term T cell activation assay by FACS measuring of CD25 activation marker on CD8+ T cells. The tested proteins, DARPin protein #2, DARPin protein #29, and DARPin protein #30 were assessed in a bispecific, T cell engager format. Such T cell engager proteins comprise a CD3-specific binding domain (SEQ ID NO: 57) in addition to the mentioned CD33-specific ankyrin repeat domains, and they are shown as DARPin protein #33, DARPin protein #31 and DARPin protein #32 respectively.

Therefore, 100,000 purified pan-T effector cells and 20,000 Molm-13 target cells per well were co-incubated (E:T ratio 5:1) with serial dilutions of selected DARPin proteins in duplicates in presence of 600 μM human serum albumin for 48 hours at 37 C. After 48 hours, cells were washed and stained with 1:1′000 Live/Dead Green (Thermo Fisher), 1:400 mouse anti-human CD8 Pacific Blue (BD), and 1:100 mouse anti-human-CD25 PerCP-Cy5.5 (eBiosciences) antibodies for 30 min at 4 C. After washing and fixation, cells were analyzed on Attune NxT (ThermoFisher) machine. T cell activation was assessed by measuring CD25+ cells on Live/Dead-negative and CD8+ gated T cells. FACS data was analyzed using FlowJo software and data was plotted using GraphPad Prism 8 (3-PL-fit). FIG. 3 shows short term T coil activation triggered by DARPin protein #33, DARPin protein #31 and DARPin protein #32 as measured by activation marker CD25. AN of the tested CD33-specific binding proteins of the invention, in a T-cell engager format, were able to bind to CD733 expressed on tumor cells and activate T-cells.

### Example 6: Assessment of Specificity and Potency of CD33-Specific Ankyrin Repeat Proteins of the Invention in T Cell Engager Format Using a Target-Specific Short-Term Tumor Cell Killing Assay

Specificity and potency of the CD33-specific ankyrin repeat proteins of the invention, DARPin protein #2, DARPin protein #29 and DARPin protein #30, In a T cell engager format (i.e. DARPin protein #33, DARPin protein #31, and DARPin protein #32 respectively), were also assessed using an in-vitro short-term cytotoxicity assay measuring LDH release. For this purpose, 100,000 purified pan-T effector cells and 20,000 Molm-13 target cells per well were co-incubated (E:T ratio 5:1) with serial dilutions of the indicated T cell engager proteins in duplicates in presence of 600 μM human serum albumin for 48 hours at 37° C. After 48 h incubation, cells were spun down and 100 μl supernatant of each well was analysed for LDH release according to manufacturer protocol (LDH detection kit; Roche Applied Science, incubation of 30 min). Absorbance was measured at 492 nm-620 nm by TECAN infinite M1000Pro reader. OD values were plotted using GraphPad Prism 5.

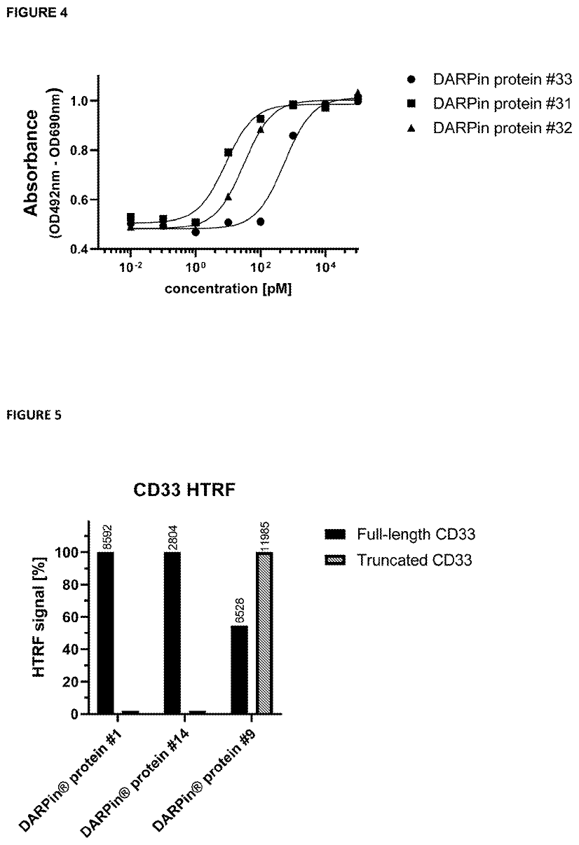

FIG. 4 shows tumor cell killing triggered by DARPin protein #33, DARPin protein #31 and DARPin protein #32. AI of the tested CD33-specific binding proteins of the invention, in a T-cell engager format, were able to bind to CD33 expressed on tumor cells and activate T-cells which in turn kill the tumor cells.

### Example 7: Determination of the CD33 Epitope Bound by CD33-Specific Binding Protein of the Invention

The CD33 epitope bound by CD33-specific ankyrin repeat proteins of the invention was investigated using HTRF and competition ELISA against the benchmark control molecule AMG330-similar (CD3-CD33 specific BiTE®, purchased from Evitria), The competition of monovalent CD33 specific binding proteins DARPin® protein #1, DARPin® protein #9 and DARPin® protein #14 and AMG330-similar for binding to human CD33 (purchased from Evitria) was assessed by HTRF and competition ELISA.

First, a binding experiment has been performed against full-length (V/C2 domains) and truncated CD33 (C2 domain) by HTRF, using crude extracts of the three CD33-specific binding proteins DARPin® protein #1. DARPin® protein #9 and DARPin® protein #14. Binding to the CD33 target was detected using Strep-Tb and anti-His-D2 HTRF reagents. In brief, crude extract of DARPi® protein #1, DARPin® protein #9 and DARPin® protein #14 was diluted 1:250 (final 1:1000) with PBSTB and added into a 384-well plate (PerkinElmer, 6008280). Then, the biotinylated target_hCD33-Fc(kih)-Avi (full-length CD33; purchased from Evitria) and hCD33-C2-Fc(kih)-Avi (truncated CD33; purchased from Evitria) respectively was diluted to 8 nM (final 6 nM) and mixed with the HTRF reagents Mab Anti-6His-d2 and Strep-Tb, both 1:200 diluted (final 1:400). As depicted in FIG. 5, all three CD33 specific binding proteins DARPin® protein #1. DARPin® protein #9 and DARPin® protein #14 show binding to the full-length CD33 but only DARPin® protein #9 binds to the truncated CD33 target. These data indicate that DARPin® protein #9 binds to the C2 domain, while DARPin® protein #91 and DARPin® protein #14 bind to the V domain of CD33.

Next, a competition ELISA experiment has been performed using CD33-binding benchmark molecule AMG330 similar. AMG330 similar, which binds to the top of the V domain as shown by Friedrch at al., 2014, was immobilized on a microplate. Biotinylated CD33 target and 50-fold excess of DARPin® protein #1, DARPin) protein #9 and DARPin® protein #14 were pre-incubated for 2 hours before binding to AMG330 was measured directly via streptavidin covalently coupled to peroxidase. In brief, a Nunc MaxiSorp 96-well plate was coated overnight with 10 nM of DARPin® protein #1, DARPin® protein #9 and DARPin® protein #14 and 5 nM of AMG330-similar. The ELISA plate was washed three times and blocked with PBSTC (PBS containing 0.1% (v/v) Tween20@ and 0.25% casein) for 4 h15 min at 450 rpm. Meanwhile, 500 nM of competitor DARPin® protein #1, DARPin® protein #9 and DARPin® protein #14 were pre-incubated with 20 nM of bio.hCD33 in a 1:1 ratio for 2 h at RT. Bio.hCD33 without competitor was included as positive control. The pre-incubated samples were then added into the coated MaxiSorp plate and incubated for 30 minutes at RT and 450 rpm. The plate was washed three times with PBST before detecting the target with a streptavidin-POD antibody (Roche, catalog number: 11 089 153 001). For detection, a freshly prepared TMB buffer (30 mM Citrate buffer pH 4.1, 5% (v/v) TMB solution (from Carl Roth GmbH) and 0.16% H2O2) was added and the reaction was stopped with 1 M H2SO4. The absorbance was measured at OD450 and referenced against OD620 using a Sunrise microplate reader (Tecan). The data were analyzed by subtracting the buffer (PBS) value. GraphPad Prism was used for analysis.

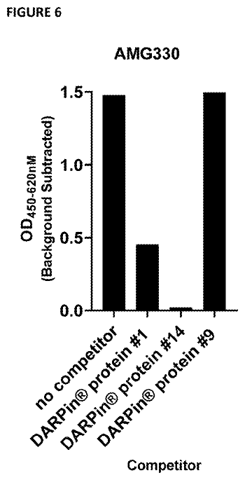

As expected. C2 domain binding DARPin® protein #9 shows no competitive binding with AMG330 similar. For both V domain binding molecules DARPin® protein #1 or DARPin® protein #14, competition resulted in full or partial inhibition of the EUSA signal, respectively. Therefore, DARPin® protein #1 binds to the V-domain and shows a partial competition with AMG330 and DARPin® protein #14. In addition, DARPin® protein #14 also binds to the V-domain and fully competes for binding with the AMG330, while DARPin® protein #9 binds to the C2 domain allowing simultaneous binding of AMG330 to CD33 (FIG. 6)

### Example 8: In Vivo Efficacy Evaluation of an Exemplary Multi-Domain TCE Binding Protein Comprising DARPin® Protein #29, in PBMC Humanized Mice and MOLM-13 Tumor Model

DARPin® protein #29, formatted in a multi-domain T-cel engager binding protein that additionally comprises two ankyrin repeat domains with binding specificity for human serum albumin, two ankyrin repeat domains with binding specificity for tumor-associated antigen 1 (TAA1) and tumor associated antigen 2 (TAA2), respectively, and one ankyrin repeat domain with binding specificity for CD3, was tested in a Peripheral Blood Mononuclear Cell (PBMC) humanized mouse model bearing the tumor cell line MOLM-13. The in vivo experiments were performed in 6 to 9-week-old female immunodeficient NXG mice (provided by Janvier Labs). Mice were maintained under standardized environment conditions in standard rodent micro-Isolator cages (20+/−1° C. room temperature, 50+/−10% relative humidity and 12 hours light dark cycle). Mice received Irradiated food and bedding and 0.22 um filtered drinking water. All experiments were done according to the Swiss Animal Protection Law with authorization from the cantonal and federal veterinary authorities.

Mice were injected intraperitonealy with hPBMC (5×10⁶ PBMC prepared from buffy-coats from two different donors) two days before the xenograft of the cancer cells. MOLM-13 cells were xenografted subcutaneously (s.c.) on the right flank area into the mice. Two hPBMC donors were used. Treatments were injected intravenously (i.v.) starting four days after cancer cell implantation. Treatments were administrated as follows:
DARPin® protein #29 in multi-domain TCE format, or vehicle, was administered i.v. three times per week for 2 weeks at 0.5 mg/kg
Tumor size was evaluated by caliper measurement. Tumor volumes were calculated using the following formula: tumor volume (mm³)=0.5×length×width².

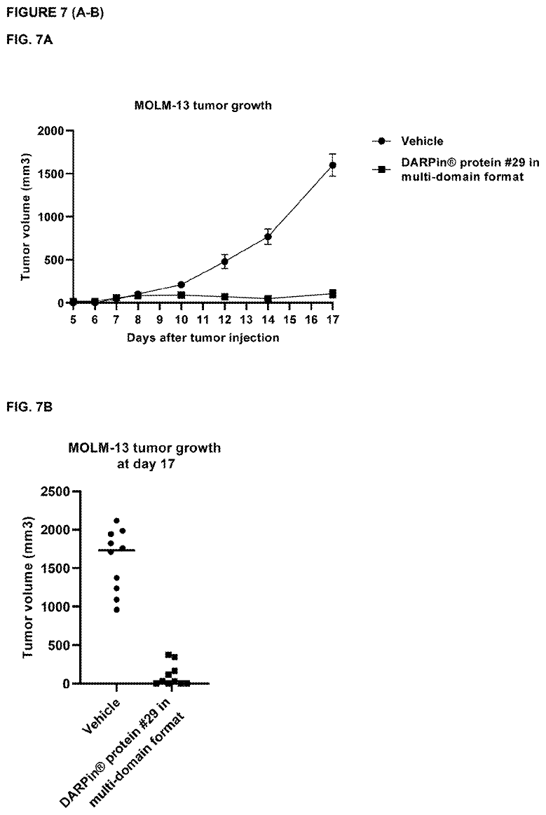

As it can be seen in FIG. 7 (A-B). DARPin® protein #29 in multi-domain TCE format shows good efficacy in terms of inhibition of tumor growth and tumor volume over the entire time of the experiment (FIG. 7A) and at 17 days after the first injection (FIG. 7B).

### Example 9: In Vitro Efficacy Evaluation of Exemplary Multi-Domain TCE Binding Proteins Comprising DARPin® Protein #29 or DARPIn® Protein #30, Using MOLM13 Wild-Type and MOLM13 CRISPR CD33 Knock-Out (KO) Target Cells in Co-Culture with Human Pan-T Coils

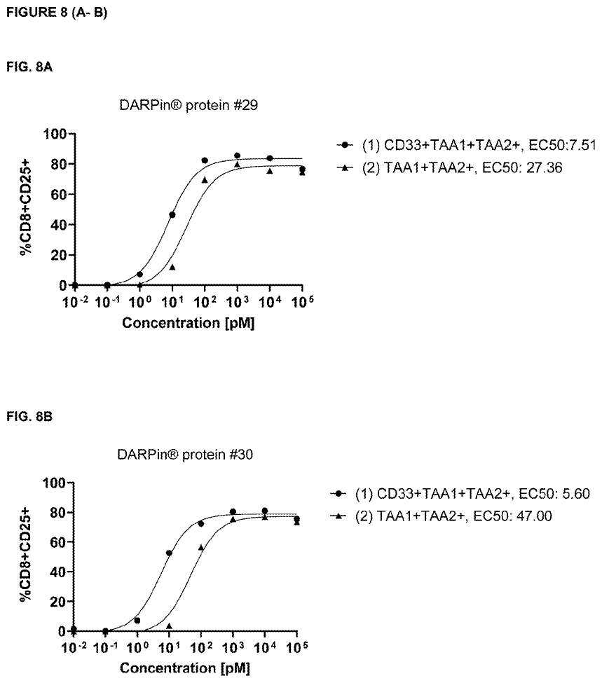

DARPin® protein #29 and DARPin® protein #30, each formatted in a multi-domain T-cel engager binding protein that additionally comprises two ankyrin repeat domains with binding specificity for human serum albumin, two ankyrin repeat domains with binding specificity for tumor-associated antigen 1 (TAA1) and tumor associated antigen 2 (TAA2), respectively, and one ankyrin repeat domain with binding specificity for CD3, were tested in an in vitro short-term T cel activation assay by FACS measuring the CD25 activation marker on CD8+ T cells. In this assay, Pan-T cells were co-cultured with target cells, whereby the target cells were either (1) Molm-13 tumor cells having wild-type target expression for CD33, TAA1 and TAA2, or (2) Molm-13 tumor cells in which expression of CD33 (but not of TAA1 and TAA2) has been eliminated by CRISPR Knock-Out (KO) technology (FIG. 8A and FIG. 8B).

For this purpose, 100,000 purified pan-T effector cells and 20,000 target cells per well were co-incubated (E:T ratio 5:1) with serial dilutions of the respective multi-domain T-cell engager binding protein in duplicates in presence of 20 μM human serum albumin for 48 hours at 37 C. After 48 hours, cells were washed and stained with 1:1,000 Live/Dead Green (Thermo Fisher), 1:400 mouse anti-human CD8 Pacific Blue (BD), and 1:100 mouse anti-human-CD25 PerCP-Cy5.5 (eBiosciences) antibodies for 30 min at 4° C. After washing and fixation, cells were analyzed on a FACS Canto II (BD) machine. T cell activation was assessed by measuring CD25+ cells on Live/Dead-negative and CD8+ gated T cells. FACS data were analyzed using FlowJo software and data were plotted using GraphPad Prism 8 (3-PL-fit).

The results demonstrate that both exemplary multi-domain T-cel engager binding proteins comprising either DARPin® protein #29 (FIG. 8A) or DARPin® protein #30 (FIG. 88) were capable of potently activating T cells in the presence of Molm-13 tumor cells with wild-type expression (curve 1; EC50 value for DARPin® protein #29 TCE: 5.71 μM; EC50 value for DARPin® protein #30 TCE: 5.80 μM).

Furthermore, the results also demonstrate that the activation of T cells was significantly reduced if the expression of CD33 in the Molm-13 tumor cells was eliminated (curve 2; EC50 value for DARPin® protein #29 TCE: 27.36 μM; EC50 value for DARPin® protein #30 TCE: 47.00 μM). This provides evidence that DARPin® protein #29 (FIG. 8A) and DARPin® protein #30 (FIG. 88) were functional in the context of the exemplary multi-domain T-cell engager binding proteins and contributed significantly to the overall potency of the multi-specific T-cell engager proteins by virtue of their ability to specifically bind to CD33 on target cells.

The specification is most thoroughly understood In light of the teachings of the references cited within the specification. The aspects within the specification provide an Illustration of aspects of the Invention and should not be construed to limit the scope of the Invention. The skilled artisan readily recognizes that many other aspects are encompassed by the invention. AN publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific aspects of the invention described herein. Such equivalents are intended to be encompassed by the following claims

SEQUENCES

| SEQ | Description for | Description | |
|---|---|---|---|
| ID | Sequence | in the patent | |
| NO | Listing | application | Sequence |
| 1 | Ankyrin repeat | DARPin ® | DLGWKLLLAASRGQDDEVRILLAAGADVNAKDIDEGYTPLHIAAYYGHLEIVEVLLKAGADVNAKD |
| | domain specific | protein #1 | RYGKTPLHLAAISGHEDIAEVLLKAGADVNAQDDKGDTPADLAADYGHEDIAEVLQKAA |
| | for CD33 | | |
| 2 | Ankyrin repeat | DARPin ® | DLGVKLLLAASRGQLDEVRILLKAGADVNAKDIDEGYTPLHIAAYYGHLEIVEVLLKAGADVNAKDR |
| | domain specific | protein #2 | YGKTPLHLAAISGHLEIVEVLLKAGADVNAQDDKGDTPADLAADYGHQDIAEVLQKAA |
| | for CD33 | | |
| 3 | Ankyrin repeat | DARPin ® | DLGVKLLRAAFHGQDDEVRILLAAGADVNAKDTDGETPLHYAAQFGHLEIVEVLLKAGADVNAKD |
| | domain specific | protein #3 | AYGATPLHWAAWHGHLEIVEVLLKAGADVNAQDVSGATPADLAAKVGHEDIAEVLQKAA |
| | for CD33 | | |
| 4 | Ankyrin repeat | DARPin ® | DLGVKLLRAAVHGQLDEVRILLKAGADVNAKDTDGETPLHYAAQFGHLEIVEVLLKAGADVNAKDA |
| | domain specific | protein #4 | YGATPLHWAAWHGHLEIVEVLLKAGADVNAQDVSGATPADLAAKVGHQDIAEVLQKAA |
| | for CD33 | | |
| 5 | Ankyrin repeat | DARPin ® | DLSKKLLQAARAGQLDEVRELLKAGADVNAKDQIGQTPLHLAAQSGHLEIVEVLLKAGADVNAKDT |
| | domain specific | protein #5 | EGWTPLHVAAWEGHLEIVEVLLKAGADVNAKDWTGETPLHLAAYQGHLEIVEVLLKAGADVNAQD |
| | for CD33 | | KSGKTPADLAARAGHQDIAEVLQKAA |
| 6 | Ankyrin repeat | DARPin ® | DLGKKLLQAARAGQLDEVRELLKAGADVNAKDQIGQTPLHLAAQSGHLEIVEVLLKAGADVNAKD |
| | domain specific | protein #6 | TEGWTPLHVAAWEGHLEIVEVLLKAGADVNAKDWTGETPLHLAAYQGHLEIVEVLLKAGADVNAQ |
| | for CD33 | | DKSGKTPADLAARAGHQDIAEVLQKAA |
| 7 | Ankyrin repeat | DARPin ® | DLGLKLLRAAYDGQDDEVRILLAAGADVNAKDWQGFTPLHYAAVLGHLEIVEVLLKAGADVNAKD |
| | domain specific | protein #7 | QEGATPLHLAALHGHLEIVEVLLKAGADVNAQDESGWTPADLAAKWGHEDIAEVLQKAA |
| | for CD33 | | |
| 8 | Ankyrin repeat | DARPin ® | DLGWKLLSAATLGQDDEVRILLAAGADVNAKDEWGKTPLHWAASTGHLEIVEVLLKAGADVNAKD |
| | domain specific | protein #8 | LWGHTPLHEAAAKGHLEIVEVLLKAGADGNAQDNIGDTPADLAAYQGHEDIAEVLQKAA |
| | for CD33 | | |
| 9 | Ankyrin repeat | DARPin ® | DLGYKLLWAASAGQDDEVRILLAAGADVNAKDKDGATPLHFAAAKGHLEIVEVLLKAGADVNAKD |
| | domain specific | protein #9 | NRGATPLHYVAAASGHLEIVEVLLKAGADVNAQDEWGNTPADLAAIYGHGDIAEVLQKAA |
| | for CD33 | | |
| 10 | Ankyrin repeat | DARPin ® | DLGYKLLWAASAGQDDEVRELLKAGADVNAKDKDGATPLHFAAAKGHLEIVEVLLKAGADVNAKD |
| | domain specific | protein #10 | NRGATPLHYAAASGHLEIVEVLLKAGADVNAQDEWGNTPADLAAIYGHGDIAEVLQKAA |
| | for CD33 | | |
| 11 | Ankyrin repeat | DARPin ® | DLGYKLLWAASAGQDDEVRELLKAGADVNAKDKDGATPLHFAAAKGHLEIVEVLLKAGADVNAKD |
| | domain specific | protein #11 | NRGATPLHYVAAASGHLEIVEVLLKAGADVNAQDEWGNTPADLAAIYGHEDIAEVLQKAA |
| | for CD33 | | |
| 12 | Ankyrin repeat | DARPin ® | DLGVKLLWAASAGQDDEVRELLKAGADVNAKDKDGATPLHFAAAKGHLEIVEVLLKAGADVNAKD |
| | domain specific | protein #12 | NRGATPLHYVAAASGHLEIVEVLLKAGADVNAQDEWGNTPADLAAIYGHGDIAEVLQKAA |
| | for CD33 | | |
| 13 | Ankyrin repeat | DARPin ® | DLGYKLLWAASAGQDDEVRELLKAGADVNAKDKDGATPLHFAAAKGHLEIVEVLLKAGADVNAKD |
| | domain specific | protein #13 | NRGATPLHYAAASGHLEIVEVLLKAGADVNAQDEWGNTPADLAAIYGHEDIAEVLQKAA |
| | for CD33 | | |
| 14 | Ankyrin repeat | DARPin ® | DLGWKLLNAAIAGQDDEVRILLAAGADVNAKDTEYGWTPLHIAASDGHLEIVEVLLKAGADVNAKD |
| | domain specific | protein #14 | YAGSTPLHAAAFVGHLEIVEVLLKAGADVNAQDHQGQTPADLAAQQGHVDIAEVLQKAA |
| | for CD33 | | |
| 15 | Ankyrin repeat | DARPin ® | DLGKKLLQAARAGQLDEVRELLKAGADVNAKDLIGWTPLHLAAHYGHLEIVEVLLKAGADVNAKD |
| | domain specific | protein #15 | AQGWTPLHIAAWEGHLEIVEVLLKAGADVNAKDWTGETPLHLAAFEGHLEIVEVLLKAGADVNAQ |
| | for CD33 | | DKSGKTPADLAARAGHQDIAEVLQKAA |
| 16 | Ankyrin repeat | DARPin ® | DLGKKLLQAARAGQLDEVRELLKAGADVNAKDHQGETPLHLAASQGHLEIVEVLLKAGADVNAKD |
| | domain specific | protein #16 | LAGYTPLHLAAWTGHLEIVEVLLKAGADVNAKDQWGRTPLHIAAASGHLEIVEVLLKAGADVNAQD |
| | for CD33 | | KSGKTPADLAARAGHQDIAEVLQKAA |
| 17 | Ankyrin repeat | DARPin ® | DLGWKLLLAASRGQDDEVRILLAAGADVNAKDIDEGYTPLHIAAYYGHLEIVEVLLKAGADVNAKD |
| | protein specific | protein #17 | RYGKTPLHLAAISGHEDIAEVLLKAGADVNAQDDKGDTPADLAADYGHEDIAEVLQKAAGSPTPTP |
| | for CD33-CD3 | | TTPTPTPTTPTPTPTGSDLGQKLLEAAWAGQDDEVRELLKAGADVNAKNSRGWTPLHTAAQTGH |
| | | | LEIFEVLLKAGADVNAKDDKGVTPLHLAAALGHLEIVEVLLKAGADVNAQDSWGTTPADLAAKYGH |
| | | | EDIAEVLQKAA |
| 18 | Ankyrin repeat | DARPin ® | DLGVKLLLAASRGQLDEVRILLKAGADVNAKDIDEGYTPLHIAAYYGHLEIVEVLLKAGADVNAKDR |
| | protein specific | protein #18 | YGKTPLHLAAISGHLEIVEVLLKAGADVNAQDDKGDTPADLAADYGHQDIAEVLQKAAGSPTPTPT |
| | for CD33-CD3 | | TPTPTPTTPTPTPTGSDLGQKLLEAAWAGQDDEVRELLKAGADVNAKNSRGWTPLHTAAQTGHL |
| | | | EIFEVLLKAGADVNAKDDKGVTPLHLAAALGHLEIVEVLLKAGADVNAQDSWGTTPADLAAKYGHE |
| | | | DIAEVLQKAA |
| 19 | Ankyrin repeat | DARPin ® | DLGVKLLRAAFHGQDDEVRILLAAGADVNAKDTDGETPLHYAAQFGHLEIVEVLLKAGADVNAKD |
| | protein specific | protein #19 | AYGATPLHWAAWHGHLEIVEVLLKAGADVNAQDVSGATPADLAAKVGHEDIAEVLQKAAGSPTPT |
| | for CD33-CD3 | | PTTPTPTPTTPTPTPTGSDLGQKLLEAAWAGQDDEVRELLKAGADVNAKNSRGWTPLHTAAQTG |
| | | | HLEIFEVLLKAGADVNAKDDKGVTPLHLAAALGHLEIVEVLLKAGADVNAQDSWGTTPADLAAKYG |
| | | | HEDIAEVLQKAA |
| 20 | Ankyrin repeat | DARPin ® | DLGVKLLRAAVHGQLDEVRILLKAGADVNAKDTDGETPLHYAAQFGHLEIVEVLLKAGADVNAKDA |
| | protein specific | protein #20 | YGATPLHWAAWHGHLEIVEVLLKAGADVNAQDVSGATPADLAAKVGHQDIAEVLQKAAGSPTPT |
| | for CD33-CD3 | | PTTPTPTPTTPTPTPTGSDLGQKLLEAAWAGQDDEVRELLKAGADVNAKNSRGWTPLHTAAQTG |
| | | | HLEIFEVLLKAGADVNAKDDKGVTPLHLAAALGHLEIVEVLLKAGADVNAQDSWGTTPADLAAKYG |
| | | | HEDIAEVLQKAA |
| 21 | Ankyrin repeat | DARPin ® | DLSKKLLQAARAGQLDEVRELLKAGADVNAKDQIGQTPLHLAAQSGHLEIVEVLLKAGADVNAKDT |
| | Ankyrin repeat | protein #21 | EGWTPLHVAAWEGHLEIVEVLLKAGADVNAKDWTGETPLHLAAYQGHLEIVEVLLKAGADVNAQD |
| | protein specific | | KSGKTPADLAARAGHQDIAEVLQKAAGSPTPTPTTPTPTPTTPTPTPTGSDLGQKLLEAAWAGQD |
| | | | DEVRELLKAGADVNAKNSRGWTPLHTAAQTGHLEIFEVLLKAGADVNAKDDKGVTPLHLAAALGH |
| | | | LEIVEVLLKAGADVNAQDSWGTTPADLAAKYGHEDIAEVLQKAA |
| 22 | Ankyrin repeat | DARPin ® | DLGKKLLQAARAGQLDEVRELLKAGADVNAKDQIGQTPLHLAAQSGHLEIVEVLLKAGADVNAKD |
| | protein specific | protein #22 | TEGWTPLHVAAWEGHLEIVEVLLKAGADVNAKDWTGETPLHLAAYQGHLEIVEVLLKAGADVNAQ |
| | for CD33-CD3 | | DKSGKTPADLAARAGHQDIAEVLQKAAGSPTPTPTTPTPTPTTPTPTPTGSDLGQKLLEAAWAGQ |
| | | | DDEVRELLKAGADVNAKNSRGWTPLHTAAQTGHLEIFEVLLKAGADVNAKDDKGVTPLHLAAALG |
| | | | HLEIVEVLLKAGADVNAQDSWGTTPADLAAKYGHEDIAEVLQKAA |
| 23 | Ankyrin repeat | DARPin ® | DLGLKLLRAAYDGQDDEVRILLAAGADVNAKDWQGFTPLHYAAVLGHLEIVEVLLKAGADVNAKD |
| | protein specific | protein #23 | QEGATPLHLAALHGHLEIVEVLLKAGADVNAQDESGWTPADLAAKWGHEDIAEVLQKAAGSPTPT |
| | for CD33-CD3 | | PTTPTPTPTTPTPTPTGSDLGQKLLEAAWAGQDDEVRILLAAGADVNAKNSRGWTPLHTAAQTG |
| | | | HLEIFEVLLKAGADVNAKNDKRVTPLHLAAALGHLEIVEVLLKAGADVNARDSWGTTPADLAAKYG |
| | | | HGDIAEVLQKLA |
| 24 | Ankyrin repeat | DARPin ® | DLGWKLLSAATLGQDDEVRILLAAGADVNAKDEWGKTPLHWAASTGHLEIVEVLLKAGADVNAKD |
| | protein specific | protein #24 | LWGHTPLHEAAAKGHLEIVEVLLKAGADGNAQDNIGDTPADLAAYQGHEDIAEVLQKAAGSPTPT |
| | for CD33-CD3 | | PTTPTPTPTTPTPTPTGSDLGQKLLEAAWAGQDDEVRILLAAGADVNAKNSRGWTPLHTAAQTG |
| | | | HLEIFEVLLKAGADVNAKNDKRVTPLHLAAALGHLEIVEVLLKAGADVNARDSWGTTPADLAAKYG |
| | | | HGDIAEVLQKLA |
| 25 | Ankyrin repeat | DARPin ® | DLGYKLLWAASAGQDDEVRILLAAGADVNAKDKDGATPLHFAAAKGHLEIVEVLLKAGADVNAKD |
| | protein specific | protein #25 | NRGATPLHYVAAASGHLEIVEVLLKAGADVNAQDEWGNTPADLAAIYGHGDIAEVLQKAAGSPTP |
| | for CD33-CD3 | | TPTTPTPTPTTPTPTPTGSDLGQKLLEAAWAGQDDEVRILLAAGADVNAKNSRGWTPLHTAAQTG |
| | | | HLEIFEVLLKAGADVNAKNDKRVTPLHLAAALGHLEIVEVLLKAGADVNARDSWGTTPADLAAKYG |
| | | | HGDIAEVLQKLA |
| 26 | Ankyrin repeat | DARPin ® | DLGWKLLNAAIAGQDDEVRILLAAGADVNAKDTEYGWTPLHIAASDGHLEIVEVLLKAGADVNAKD |
| | protein specific | protein #26 | YAGSTPLHAAAFVGHLEIVEVLLKAGADVNAQDHQGQTPADLAAQQGHVDIAEVLQKAAGSPTPT |
| | for CD33-CD3 | | PTTPTPTPTTPTPTPTGSDLGQKLLEAAWAGQDDEVRILLAAGADVNAKNSRGWTPLHTAAQTG |
| | | | HLEIFEVLLKAGADVNAKNDKRVTPLHLAAALGHLEIVEVLLKAGADVNARDSWGTTPADLAAKYG |
| | | | HGDIAEVLQKLA |
| 27 | Ankyrin repeat | DARPin ® | DLGKKLLQAARAGQLDEVRELLKAGADVNAKDLIGWTPLHLAAHYGHLEIVEVLLKAGADVNAKD |
| | protein specific | protein #27 | AQGWTPLHIAAWEGHLEIVEVLLKAGADVNAKDWTGETPLHLAAFEGHLEIVEVLLKAGADVNAQ |
| | for CD33-CD3 | | DKSGKTPADLAARAGHQDIAEVLQKAAGSPTPTPTTPTPTPTTPTPTPTGSDLGQKLLEAAWAGQ |
| | | | DDEVRILLAAGADVNAKNSRGWTPLHTAAQTGHLEIFEVLLKAGADVNAKNDKRVTPLHLAAALG |
| | | | HLEIVEVLLKAGADVNARDSWGTTPADLAAKYGHGDIAEVLQKLA |
| 28 | Ankyrin repeat | DARPin ® | DLGKKLLQAARAGQLDEVRELLKAGADVNAKDHQGETPLHLAASQGHLEIVEVLLKAGADVNAKD |
| | protein specific | protein #28 | LAGYTPLHLAAWTGHLEIVEVLLKAGADVNAKDQWGRTPLHIAAASGHLEIVEVLLKAGADVNAQD |
| | for CD33-CD3 | | KSGKTPADLAARAGHQDIAEVLQKAAGSPTPTPTTPTPTPTTPTPTPTGSDLGQKLLEAAWAGQD |
| | | | DEVRILLAAGADVNAKNSRGWTPLHTAAQTGHLEIFEVLLKAGADVNAKNDKRVTPLHLAAALGHL |
| | | | EIVEVLLKAGADVNARDSWGTTPADLAAKYGHGDIAEVLQKLA |
| 29 | Ankyrin repeat | | KDIDEGYTPLHIAAYYGHLEIVEVLLKAGADVNA |
| | module | | |
| 30 | Ankyrin repeat | | KDRYGKTPLHLAAISGHEDIAEVLLKAGADVNA |
| | module | | |
| 31 | Ankyrin repeat | | KDRYGKTPLHLAAISGHLEIVEVLLKAGADVNA |
| | module | | |
| 32 | Ankyrin repeat | | KDTDGETPLHYAAQFGHLEIVEVLLKAGADVNA |
| | module | | |
| 33 | Ankyrin repeat | | KDAYGATPLHWAAWHGHLEIVEVLLKAGADVNA |
| | module | | |
| 34 | Ankyrin repeat | | KDQIGQTPLHLAAQSGHLEIVEVLLKAGADVNA |
| | module | | |
| 35 | Ankyrin repeat | | KDTEGWTPLHVAAWEGHLEIVEVLLKAGADVNA |
| | module | | |
| 36 | Ankyrin repeat | | KDWTGETPLHLAAYQGHLEIVEVLLKAGADVNA |
| | module | | |
| 37 | Ankyrin repeat | | KDWQGFTPLHYAAVLGHLEIVEVLLKAGADVNA |
| | module | | |
| 38 | Ankyrin repeat | | KDQEGATPLHLAALHGHLEIVEVLLKAGADVNA |
| | module | | |
| 39 | Ankyrin repeat | | KDEWGKTPLHWAASTGHLEIVEVLLKAGADVNA |
| | module | | |
| 40 | Ankyrin repeat | | KDLWGHTPLHEAAAKGHLEIVEVLLKAGADGNA |
| | module | | |
| 41 | Ankyrin repeat | | KDKDGATPLHFAAAKGHLEIVEVLLKAGADVNA |
| | module | | |
| 42 | Ankyrin repeat | | KDNRGATPLHYVAAASGHLEIVEVLLKAGADVNA |
| | module | | |
| 43 | Ankyrin repeat | | KDNRGATPLHYAAASGHLEIVEVLLKAGADVNA |
| | module | | |
| 44 | Ankyrin repeat | | KDTEYGWTPLHIAASDGHLEIVEVLLKAGADVNA |
| | module | | |
| 45 | Ankyrin repeat | | KDYAGSTPLHAAAFVGHLEIVEVLLKAGADVNA |
| | module | | |
| 46 | Ankyrin repeat | | KDLIGWTPLHLAAHYGHLEIVEVLLKAGADVNA |
| | module | | |
| 47 | Ankyrin repeat | | KDAQGWTPLHIAAWEGHLEIVEVLLKAGADVNA |
| | module | | |
| 48 | Ankyrin repeat | | KDWTGETPLHLAAFEGHLEIVEVLLKAGADVNA |
| | module | | |
| 49 | Ankyrin repeat | | KDHQGETPLHLAASQGHLEIVEVLLKAGADVNA |
| | module | | |
| 50 | Ankyrin repeat | | KDLAGYTPLHLAAWTGHLEIVEVLLKAGADVNA |
| | module | | |
| 51 | Ankyrin repeat | | KDQWGRTPLHIAAASGHLEIVEVLLKAGADVNA |
| | module | | |
| 52 | Ankyrin repeat | | DLGKKLLEAARAGQDDEVRELLKAGADVNAKDYFSHTPLHLAARNGHLKIVEVLLKAGADVNAKD |
| | domain specific | | FAGKTPLHLAANEGHLEIVEVLLKAGADVNAQDIFGKTPADIAADAGHEDIAEVLQKAA |
| | for human serum | | |
| | albumin | | |
| 53 | Ankyrin repeat | | DLGKKLLEAARAGQDDEVRELLKAGADVNAKDYFSHTPLHLAARNGHLKIVEVLLKAGADVNAKD |
| | domain specific | | FAGKTPLHLAAADGHLEIVEVLLKAGADVNAQDIFGKTPADIAADAGHEDIAEVLQKAA |
| | for human serum | | |
| | albumin | | |
| 54 | Ankyrin repeat | | DLGKKLLEAARAGQDDEVRELLKAGADVNAKDYFSHTPLHLAARNGHLKIVEVLLKAGADVNAKD |
| | domain specific | | FAGKTPLHLAADAGHLEIVEVLLKAGADVNAQDIFGKTPADIAADAGHEDIAEVLQKAA |
| | for human serum | | |
| | albumin | | |
| 55 | Ankyrin repeat | | DLGQKLLEAAWAGQDDEVRELLKAGADVNAKDSQGWTPLHTAAQTGHLEIFEVLLKAGADVNAK |
| | domain specific | | DDKGVTPLHLAAALGHLEIVEVLLKAGADVNAQDSWGTTPADLAAKYGHEDIAEVLQKAA |
| | for CD3 | | |
| 56 | Ankyrin repeat | | DLGQKLLEAAWAGQDDEVRELLKAGADVNAKNSRGWTPLHTAAQTGHLEIFEVLLKAGADVNAK |
| | domain specific | | DDKGVTPLHLAAALGHLEIVEVLLKAGADVNAQDSWGTTPADLAAKYGHEDIAEVLQKAA |
| | for CD3 | | |
| 57 | Ankyrin repeat | | DLGQKLLEAAWAGQDDEVRELLKAGADVNAKNSRGWTPLHTAAQTGHLEIFEVLLKAGADVNAK |
| | domain specific | | NDKRVTPLHLAAALGHLEIVEVLLKAGADVNARDSWGTTPADLAAKYGHQDIAEVLQKAA |
| | for CD3 | | |
| 58 | Ankyrin repeat | | DLGQKLLEAAWAGQLDEVRILLKAGADVNAKNSRGWTPLHTAAQTGHLEIFEVLLKAGADVNAKT |
| | domain specific | | NKRVTPLHLAAALGHLEIVEVLLKAGADVNARDTWGTTPADLAAKYGHRDIAEVLQKAA |
| | for CD3 | | |
| 59 | Ankyrin repeat | | DLGQKLLEAAWAGQDDEVRILLAAGADVNAKNSRGWTPLHTAAQTGHLEIFEVLLKAGADVNAKN |
| | domain specific | | DKRVTPLHLAAALGHLEIVEVLLKAGADVNARDSWGTTPADLAAKYGHGDIAEVLQKLN |
| | for CD3 | | |
| 60 | CD33 target | | DPNFWLQVQESVTVQEGLCVLVPCTFFHPIPYYDKNSPVHGYWFREGAIISGDSPVATNKLDQEV |
| | protein (ECD) | | QEETQGRFRLLGDPSRNNCSLSIVDARRRDNGSYFFRMERGSTKYSYKSPQLSVHVTDLTHRPKI |
| | | | LIPGTLEPGHSKNLTCSVSWACEQGTPPIFSWLSAAPTSLGPRTTHSSVLIITPRPQDHGTNLTCQ |
| | | | VKFAGAGVTTERTIQLNVTYVPQNPTTGIFPGDGSGKQETRAGVVH |
| 61 | CD33 target | | DLTHRPKILIPGTLEPGHSKNLTCSVSWASEQGTPPIFSWLSAAPTSLGPRTTHSSVLIITPRPQDH |
| | protein (ECD | | GTNLTCQVKFAGAGVTTERTIQLNVTYVPQNPTTGIFPGDGSGKQETRAGVVH |
| | splice variant) | | |
| 62 | Ankyrin repeat | | xDxxGxTPLHLAxxxGxxxIVxVLLxxGADVNA |
| | module | | wherein ″x″ denotes any amino acid (preferably not cysteine, |
| | | | glycine, or proline) |
| 63 | Ankyrin repeat | | xDxxGxTPLHLAAxxGHLEIVEVLLKxGADVNA |
| | module | | wherein ″x″ in positions 1, 3, 4, 6, 14, 15 denotes any amino acid |
| | | | (preferably not cysteine, glycine, or proline), and ″x″ in position |
| | | | 27 is selected from the group consisting of asparagine, histidine, |
| | | | or tyrosine |
| 64 | Ankyrin repeat | | GSDLGWKLLLAASRGQDDEVRILLAAGADVNAKDIDEGYTPLHIAAYYGHLEIVEVLLKAGADVNA |
| | domain specific | | KDRYGKTPLHLAAISGHEDIAEVLLKAGADVNAQDDKGDTPADLAADYGHEDIAEVLQKAA |
| | for CD33 | | |
| 65 | PT-rich peptide | | GSPTPTPTTPTPTPTTPTPTPTGS |
| | linker | | |
| 66 | PT-rich peptide | | GSPTPTPTTPTPTPTTPTPTPT |
| | linker | | |
| 67 | Consensus GS | | [Gly-Gly-Gly-Gly-Ser]n, wherein n is 1, 2, 3, 4, 5, or 6 |
| | linker | | |
| 68 | His-tag | | MRGSHHHHHH |
| 69 | N-cap | | DLGKKLLQAARAGQLDEVRELLKAGADVNA |
| 70 | N-cap | | DLGKKLLQAARAGQLDEVRILLKAGADVNA |
| 71 | N-cap | | DLGKKLLQAARAGQLDEVRILLAAGADVNA |
| 72 | N-Cap | | DLGXXLLQAAXXGQLDXVRXLXXXGADVNA |
| | (randomized) | | wherein ″X″ denotes any amino acid (preferably not cysteine, |
| | | | glycine, or proline) |
| 73 | C-cap | | QDKFGKTPADIAADNGHEDIAEVLQKLN |
| 74 | C-cap | | QDKSGKTPADLAARAGHQDIAEVLQKAA |
| 75 | C-cap | | QDSSGFTPADLAALVGHEDIAEVLQKLA |
| 76 | C-cap | | XDXXGXTPXXXAARXGXQXXXXXXXXAA |
| | (randomized) | | wherein ″X″ denotes any amino acid (preferably not cysteine, |
| | | | glycine, or proline) |
| 77 | Ankyrin repeat | DARPin ® | DLGVKLLLAAERGQLDEVRILLKAGADVNAKDIAEGYTPLHIAAYQGHLEIVEVLLKAGADVNAKDR |
| | domain specific | protein #29 | YGKTPLHLAAIGGHLEIVEVLLKAGADVNAQDNKGSTPADLAADYGHQDIAEVLQKAA |
| | for CD33 | | |
| 78 | Ankyrin repeat | DARPin ® | DLGKKLLLAAERGQLDEVRILLKAGADVNAKDRAEGYTPLHIAAYQGHLEIVEVLLKAGADVNAKD |
| | domain specific | protein #30 | RYGKTPLHLAAISGHLEIVEVLLKAGADVNAQDNKGSTPADLAADYGHQDIAEVLQKAA |
| | for CD33 | | |
| 79 | Ankyrin repeat | | KDIAEGYTPLHIAAYQGHLEIVEVLLKAGADVNA |
| | module | | |
| 80 | Ankyrin repeat | | KDRYGKTPLHLAAIGGHLEIVEVLLKAGADVNA |
| | module | | |
| 81 | Ankyrin repeat | | KDRAEGYTPLHIAAYQGHLEIVEVLLKAGADVNA |
| | module | | |
| 82 | Ankyrin repeat | DARPin ® | DLGVKLLLAAERGQLDEVRILLKAGADVNAKDIAEGYTPLHIAAYQGHLEIVEVLLKAGADVNAKDR |
| | protein specific | protein #31 | YGKTPLHLAAIGGHLEIVEVLLKAGADVNAQDNKGSTPADLAADYGHQDIAEVLQKAAGSPTPTPT |
| | for CD33-CD3 | | TPTPTPTTPTPTPTGSDLGQKLLEAAWAGQDDEVRELLKAGADVNAKNSRGWTPLHTAAQTGHL |
| | | | EIFEVLLKAGADVNAKNDKRVTPLHLAAALGHLEIVEVLLKAGADVNARDSWGTTPADLAAKYGHQ |
| | | | DIAEVLQKAA |
| 83 | Ankyrin repeat | DARPin ® | DLGKKLLLAAERGQLDEVRILLKAGADVNAKDRAEGYTPLHIAAYQGHLEIVEVLLKAGADVNAKD |
| | protein specific | protein #32 | RYGKTPLHLAAISGHLEIVEVLLKAGADVNAQDNKGSTPADLAADYGHQDIAEVLQKAAGSPTPTP |
| | for CD33-CD3 | | TTPTPTPTTPTPTPTGSDLGQKLLEAAWAGQDDEVRELLKAGADVNAKNSRGWTPLHTAAQTGH |
| | | | LEIFEVLLKAGADVNAKNDKRVTPLHLAAALGHLEIVEVLLKAGADVNARDSWGTTPADLAAKYGH |
| | | | QDIAEVLQKAA |
| 84 | Ankyrin repeat | DARPin ® | DLGVKLLLAASRGQLDEVRILLKAGADVNAKDIDEGYTPLHIAAYYGHLEIVEVLLKAGADVNAKDR |
| | protein specific | protein #33 | YGKTPLHLAAISGHLEIVEVLLKAGADVNAQDDKGDTPADLAADYGHQDIAEVLQKAAGSPTPTPT |
| | for CD33-CD3 | | TPTPTPTTPTPTPTGSDLGQKLLEAAWAGQDDEVRELLKAGADVNAKNSRGWTPLHTAAQTGHL |
| | | | EIFEVLLKAGADVNAKNDKRVTPLHLAAALGHLEIVEVLLKAGADVNARDSWGTTPADLAAKYGHQ |
| | | | DIAEVLQKAA |
| 85 | His-tag | | MRGSHHHHHHGS |

1. A recombinant binding protein comprising an ankyrin repeat domain, wherein said ankyrin repeat domain has binding specificity for human CD33, and wherein said ankyrin repeat domain comprises an ankyrin repeat module having an amino acid sequence selected from the group consisting of (1) SEQ ID NOs: 29 to 51 and 79 to 81, and (2) sequences in which up to 9 amino acids in any of SEQ ID NOS: 29 to 51 and 79 to 81 are substituted by another amino acid.

2. A recombinant binding protein comprising an ankyrin repeat domain, wherein said ankyrin repeat domain has binding specificity for human CD33, and wherein said ankyrin repeat domain comprises an amino acid sequence with at least 85% amino acid sequence identity with any one of SEQ ID NOs: 1 to 16 and 77 to 78.

3. The recombinant binding protein of any preceding claim, wherein said ankyrin repeat domain binds human CD33 in PBS with a dissociation constant (K_{D}) below about 100 nM, optionally with a K_{D} between about 0.1 nM and about 100 nM.

4. The recombinant binding protein of any preceding claim, wherein said ankyrin repeat domain binds human CD33 with an EC₅₀ ranging from about from about 0.1 nM to about 10 nM.

5. The recombinant binding protein of any preceding claim, further comprising a binding moiety with binding specificity for a target expressed on an immune cel.

6. The recombinant binding protein of claim 5, wherein said immune el is a T cel and wherein said target expressed on an immune cell is CD3.

7. The recombinant binding protein of any one of claims 5 to 6, wherein said binding moiety with binding specificity for a target expressed on an immune cell is an ankyrin repeat domain.

8. The recombinant binding protein of any of claims 5 to 7, wherein said binding moiety with binding specificity for a target expressed on an immune cell is an ankyrin repeat domain with binding specificity for human CD3.

9. The recombinant binding protein of any of claims 5 to 7, wherein said binding moiety with binding specificity for a target expressed on an immune cell is an ankyrin repeat domain with binding specificity for human CD3, and wherein said ankyrin repeat domain with binding specificity for human CD3 comprises an amino acid sequence that is at least 85% amino acid sequence Identity with any one of SEQ ID NOS: 55 to 59.

10. The recombinant binding protein of claim 9, wherein said ankyrin repeat domain with binding specificity for human CD3 comprises the amino acid sequence of any one of SEQ ID NOs: 55 to 59.

11. The recombinant binding protein of any of claims 5 to 10, wherein said ankyrin repeat domain with binding specificity for human CD33 and said binding moiety with binding specifity for a target expressed on an immune cl are covalently inked with a peptide inker.

12. The recombinant binding protein of claim 11, wherein said peptide linker is a proline-threonine-rich peptide linker.

13. The recombinant binding protein of claims 11 to 12, wherein the amino acid sequence of said peptide linker has a length from 1 to 50 amino acids.

14. The recombinant binding protein of any preceding claim, wherein said binding protein further comprises a half-life extending moiety.

15. The recombinant binding protein of claim 14, wherein said half-life extending moiety is an ankyrin repeat domain with binding specificity for human serum albumin.

16. The recombinant binding protein of claim 15, wherein said ankyrin repeat domain with binding specificity for human serum albumin comprises an amino acid sequence that is at least 85% identical to the amino acid sequence of any one of SEQ ID NOs: 52 to 54.

17. The recombinant binding protein of claims 11 and 12, wherein said ankyrin repeat domain with binding specificity for human serum albumin comprises the amino acid sequence of any one of SEQ ID NOs: 52 to 54.

18. The recombinant binding protein of any of the preceding claims, wherein said binding protein further comprises at least one binding moiety with binding specificity for a target expressed in a tumor cell, wherein said target expressed in a tumor cell is different from human CD33.

19. A nucleic acid encoding the recombinant binding protein of any of the preceding claims.

20. A pharmaceutical composition comprising the recombinant binding protein of any of claims 1 to 18 or the nucleic acid of claim 19, and a pharmaceutically acceptable carrier and/or diluent.

21. A method of immune call activation in a tumor tissue of a human patient, the method comprising the step of administering to said patient the recombinant binding protein of any one of claims 1 to 18, the nucleic acid of claim 19, or the pharmaceutical composition of claim 20.

22. The method of claim 21, wherein said immune cell is a T cell.

23. A method of treating a medical condition, the method comprising the step of administering to a patient in need thereof a therapeutically effective amount of the recombinant binding protein of any one of claims 1 to 18, the nucleic acid of claim 19, or the pharmaceutical composition of claim 20.

24. The method of claim 23, wherein said medical condition is a cancer.

25. The method of claim 23, wherein said medical condition is a cancer characterized by a liquid tumor.

26. The method of claim 23, wherein said medical condition is leukemia.

27. The method of claim 23, wherein said medical condition is acute myeloid leukemia.

28. The recombinant binding protein of any one of claims 1 to 18, the nucleic acid of claim 19, or the pharmaceutical composition of claim 20, for use in therapy.

29. The recombinant binding protein of any one of claims 1 to 18, the nucleic acid of claim 19, or the pharmaceutical composition of claim 20, for use in treating cancer, optionally for use in treating a cancer characterized by a liquid tumor.

30. The recombinant binding protein or the pharmaceutical composition for use according to claim 29, wherein said cancer is leukemia, optionally wherein said cancer is acute myeloid leukemia.


SEQUENCE LISTING (85 sequences)

SEQ ID NO: 1 - protein, 125 aa, Artificial Sequence
  DARPin protein #1
DLGWKLLLAASRGQDDEVRILLAAGADVNAKDIDEGYTPLHIAAYYGHLEIVEVLLKAGADVNAKDRYGKTPLHLAAISGHEDIAEVLLKAGADVNAQDDKGDTPADLAADYGHEDIAEVLQKAA

SEQ ID NO: 2 - protein, 125 aa, Artificial Sequence
  DARPin protein #2
DLGVKLLLAASRGQLDEVRILLKAGADVNAKDIDEGYTPLHIAAYYGHLEIVEVLLKAGADVNAKDRYGKTPLHLAAISGHLEIVEVLLKAGADVNAQDDKGDTPADLAADYGHQDIAEVLQKAA

SEQ ID NO: 3 - protein, 124 aa, Artificial Sequence
  DARPin protein #3
DLGVKLLRAAFHGQDDEVRILLAAGADVNAKDTDGETPLHYAAQFGHLEIVEVLLKAGADVNAKDAYGATPLHWAAWHGHLEIVEVLLKAGADVNAQDVSGATPADLAAKVGHEDIAEVLQKAA

SEQ ID NO: 4 - protein, 124 aa, Artificial Sequence
  DARPin protein #4
DLGVKLLRAAVHGQLDEVRILLKAGADVNAKDTDGETPLHYAAQFGHLEIVEVLLKAGADVNAKDAYGATPLHWAAWHGHLEIVEVLLKAGADVNAQDVSGATPADLAAKVGHQDIAEVLQKAA

SEQ ID NO: 5 - protein, 157 aa, Artificial Sequence
  DARPin protein #5
DLSKKLLQAARAGQLDEVRELLKAGADVNAKDQIGQTPLHLAAQSGHLEIVEVLLKAGADVNAKDTEGWTPLHVAAWEGHLEIVEVLLKAGADVNAKDWTGETPLHLAAYQGHLEIVEVLLKAGADVNAQDKSGKTPADLAARAGHQDIAEVLQKAA

SEQ ID NO: 6 - protein, 157 aa, Artificial Sequence
  DARPin protein #6
DLGKKLLQAARAGQLDEVRELLKAGADVNAKDQIGQTPLHLAAQSGHLEIVEVLLKAGADVNAKDTEGWTPLHVAAWEGHLEIVEVLLKAGADVNAKDWTGETPLHLAAYQGHLEIVEVLLKAGADVNAQDKSGKTPADLAARAGHQDIAEVLQKAA

SEQ ID NO: 7 - protein, 124 aa, Artificial Sequence
  DARPin protein #7
DLGLKLLRAAYDGQDDEVRILLAAGADVNAKDWQGFTPLHYAAVLGHLEIVEVLLKAGADVNAKDQEGATPLHLAALHGHLEIVEVLLKAGADVNAQDESGWTPADLAAKWGHEDIAEVLQKAA

SEQ ID NO: 8 - protein, 124 aa, Artificial Sequence
  DARPin protein #8
DLGWKLLSAATLGQDDEVRILLAAGADVNAKDEWGKTPLHWAASTGHLEIVEVLLKAGADVNAKDLWGHTPLHEAAAKGHLEIVEVLLKAGADGNAQDNIGDTPADLAAYQGHEDIAEVLQKAA

SEQ ID NO: 9 - protein, 125 aa, Artificial Sequence
  DARPin protein #9
DLGYKLLWAASAGQDDEVRILLAAGADVNAKDKDGATPLHFAAAKGHLEIVEVLLKAGADVNAKDNRGATPLHYVAAASGHLEIVEVLLKAGADVNAQDEWGNTPADLAAIYGHGDIAEVLQKAA

SEQ ID NO: 10 - protein, 124 aa, Artificial Sequence
  DARPin protein #10
DLGYKLLWAASAGQDDEVRELLKAGADVNAKDKDGATPLHFAAAKGHLEIVEVLLKAGADVNAKDNRGATPLHYAAASGHLEIVEVLLKAGADVNAQDEWGNTPADLAAIYGHGDIAEVLQKAA

SEQ ID NO: 11 - protein, 125 aa, Artificial Sequence
  DARPin protein #11
DLGYKLLWAASAGQDDEVRELLKAGADVNAKDKDGATPLHFAAAKGHLEIVEVLLKAGADVNAKDNRGATPLHYVAAASGHLEIVEVLLKAGADVNAQDEWGNTPADLAAIYGHEDIAEVLQKAA

SEQ ID NO: 12 - protein, 125 aa, Artificial Sequence
  DARPin protein #12
DLGVKLLWAASAGQDDEVRELLKAGADVNAKDKDGATPLHFAAAKGHLEIVEVLLKAGADVNAKDNRGATPLHYVAAASGHLEIVEVLLKAGADVNAQDEWGNTPADLAAIYGHGDIAEVLQKAA

SEQ ID NO: 13 - protein, 124 aa, Artificial Sequence
  DARPin protein #13
DLGYKLLWAASAGQDDEVRELLKAGADVNAKDKDGATPLHFAAAKGHLEIVEVLLKAGADVNAKDNRGATPLHYAAASGHLEIVEVLLKAGADVNAQDEWGNTPADLAAIYGHEDIAEVLQKAA

SEQ ID NO: 14 - protein, 125 aa, Artificial Sequence
  DARPin protein #14
DLGWKLLNAAIAGQDDEVRILLAAGADVNAKDTEYGWTPLHIAASDGHLEIVEVLLKAGADVNAKDYAGSTPLHAAAFVGHLEIVEVLLKAGADVNAQDHQGQTPADLAAQQGHVDIAEVLQKAA

SEQ ID NO: 15 - protein, 157 aa, Artificial Sequence
  DARPin protein #15
DLGKKLLQAARAGQLDEVRELLKAGADVNAKDLIGWTPLHLAAHYGHLEIVEVLLKAGADVNAKDAQGWTPLHIAAWEGHLEIVEVLLKAGADVNAKDWTGETPLHLAAFEGHLEIVEVLLKAGADVNAQDKSGKTPADLAARAGHQDIAEVLQKAA

SEQ ID NO: 16 - protein, 157 aa, Artificial Sequence
  DARPin protein #16
DLGKKLLQAARAGQLDEVRELLKAGADVNAKDHQGETPLHLAASQGHLEIVEVLLKAGADVNAKDLAGYTPLHLAAWTGHLEIVEVLLKAGADVNAKDQWGRTPLHIAAASGHLEIVEVLLKAGADVNAQDKSGKTPADLAARAGHQDIAEVLQKAA

SEQ ID NO: 17 - protein, 249 aa, Artificial Sequence
  DARPin protein #17
DLGWKLLLAASRGQDDEVRILLAAGADVNAKDIDEGYTPLHIAAYYGHLEIVEVLLKAGADVNAKDRYGKTPLHLAAISGHEDIAEVLLKAGADVNAQDDKGDTPADLAADYGHEDIAEVLQKAADLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 18 - protein, 249 aa, Artificial Sequence
  DARPin protein #18
DLGVKLLLAASRGQLDEVRILLKAGADVNAKDIDEGYTPLHIAAYYGHLEIVEVLLKAGADVNAKDRYGKTPLHLAAISGHLEIVEVLLKAGADVNAQDDKGDTPADLAADYGHQDIAEVLQKAADLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 19 - protein, 248 aa, Artificial Sequence
  DARPin protein #19
DLGVKLLRAAFHGQDDEVRILLAAGADVNAKDTDGETPLHYAAQFGHLEIVEVLLKAGADVNAKDAYGATPLHWAAWHGHLEIVEVLLKAGADVNAQDVSGATPADLAAKVGHEDIAEVLQKAADLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 20 - protein, 248 aa, Artificial Sequence
  DARPin protein #20
DLGVKLLRAAVHGQLDEVRILLKAGADVNAKDTDGETPLHYAAQFGHLEIVEVLLKAGADVNAKDAYGATPLHWAAWHGHLEIVEVLLKAGADVNAQDVSGATPADLAAKVGHQDIAEVLQKAADLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 21 - protein, 281 aa, Artificial Sequence
  DARPin protein #21
DLSKKLLQAARAGQLDEVRELLKAGADVNAKDQIGQTPLHLAAQSGHLEIVEVLLKAGADVNAKDTEGWTPLHVAAWEGHLEIVEVLLKAGADVNAKDWTGETPLHLAAYQGHLEIVEVLLKAGADVNAQDKSGKTPADLAARAGHQDIAEVLQKAADLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 22 - protein, 281 aa, Artificial Sequence
  DARPin protein #22
DLGKKLLQAARAGQLDEVRELLKAGADVNAKDQIGQTPLHLAAQSGHLEIVEVLLKAGADVNAKDTEGWTPLHVAAWEGHLEIVEVLLKAGADVNAKDWTGETPLHLAAYQGHLEIVEVLLKAGADVNAQDKSGKTPADLAARAGHQDIAEVLQKAADLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 23 - protein, 272 aa, Artificial Sequence
  DARPin protein #23
DLGLKLLRAAYDGQDDEVRILLAAGADVNAKDWQGFTPLHYAAVLGHLEIVEVLLKAGADVNAKDQEGATPLHLAALHGHLEIVEVLLKAGADVNAQDESGWTPADLAAKWGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLA

SEQ ID NO: 24 - protein, 272 aa, Artificial Sequence
  DARPin protein #24
DLGWKLLSAATLGQDDEVRILLAAGADVNAKDEWGKTPLHWAASTGHLEIVEVLLKAGADVNAKDLWGHTPLHEAAAKGHLEIVEVLLKAGADGNAQDNIGDTPADLAAYQGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLA

SEQ ID NO: 25 - protein, 273 aa, Artificial Sequence
  DARPin protein #25
DLGYKLLWAASAGQDDEVRILLAAGADVNAKDKDGATPLHFAAAKGHLEIVEVLLKAGADVNAKDNRGATPLHYVAAASGHLEIVEVLLKAGADVNAQDEWGNTPADLAAIYGHGDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLA

SEQ ID NO: 26 - protein, 273 aa, Artificial Sequence
  DARPin protein #26
DLGWKLLNAAIAGQDDEVRILLAAGADVNAKDTEYGWTPLHIAASDGHLEIVEVLLKAGADVNAKDYAGSTPLHAAAFVGHLEIVEVLLKAGADVNAQDHQGQTPADLAAQQGHVDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLA

SEQ ID NO: 27 - protein, 305 aa, Artificial Sequence
  DARPin protein #27
DLGKKLLQAARAGQLDEVRELLKAGADVNAKDLIGWTPLHLAAHYGHLEIVEVLLKAGADVNAKDAQGWTPLHIAAWEGHLEIVEVLLKAGADVNAKDWTGETPLHLAAFEGHLEIVEVLLKAGADVNAQDKSGKTPADLAARAGHQDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLA

SEQ ID NO: 28 - protein, 305 aa, Artificial Sequence
  DARPin protein #28
DLGKKLLQAARAGQLDEVRELLKAGADVNAKDHQGETPLHLAASQGHLEIVEVLLKAGADVNAKDLAGYTPLHLAAWTGHLEIVEVLLKAGADVNAKDQWGRTPLHIAAASGHLEIVEVLLKAGADVNAQDKSGKTPADLAARAGHQDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLA

SEQ ID NO: 29 - protein, 34 aa, Artificial Sequence
  Ankyrin repeat module
KDIDEGYTPLHIAAYYGHLEIVEVLLKAGADVNA

SEQ ID NO: 30 - protein, 33 aa, Artificial Sequence
  Ankyrin repeat module
KDRYGKTPLHLAAISGHEDIAEVLLKAGADVNA

SEQ ID NO: 31 - protein, 33 aa, Artificial Sequence
  Ankyrin repeat module
KDRYGKTPLHLAAISGHLEIVEVLLKAGADVNA

SEQ ID NO: 32 - protein, 33 aa, Artificial Sequence
  Ankyrin repeat module
KDTDGETPLHYAAQFGHLEIVEVLLKAGADVNA

SEQ ID NO: 33 - protein, 33 aa, Artificial Sequence
  Ankyrin repeat module
KDAYGATPLHWAAWHGHLEIVEVLLKAGADVNA

SEQ ID NO: 34 - protein, 33 aa, Artificial Sequence
  Ankyrin repeat module
KDQIGQTPLHLAAQSGHLEIVEVLLKAGADVNA

SEQ ID NO: 35 - protein, 33 aa, Artificial Sequence
  Ankyrin repeat module
KDTEGWTPLHVAAWEGHLEIVEVLLKAGADVNA

SEQ ID NO: 36 - protein, 33 aa, Artificial Sequence
  Ankyrin repeat module
KDWTGETPLHLAAYQGHLEIVEVLLKAGADVNA

SEQ ID NO: 37 - protein, 33 aa, Artificial Sequence
  Ankyrin repeat module
KDWQGFTPLHYAAVLGHLEIVEVLLKAGADVNA

SEQ ID NO: 38 - protein, 33 aa, Artificial Sequence
  Ankyrin repeat module
KDQEGATPLHLAALHGHLEIVEVLLKAGADVNA

SEQ ID NO: 39 - protein, 33 aa, Artificial Sequence
  Ankyrin repeat module
KDEWGKTPLHWAASTGHLEIVEVLLKAGADVNA

SEQ ID NO: 40 - protein, 33 aa, Artificial Sequence
  Ankyrin repeat module
KDLWGHTPLHEAAAKGHLEIVEVLLKAGADGNA

SEQ ID NO: 41 - protein, 33 aa, Artificial Sequence
  Ankyrin repeat module
KDKDGATPLHFAAAKGHLEIVEVLLKAGADVNA

SEQ ID NO: 42 - protein, 34 aa, Artificial Sequence
  Ankyrin repeat module
KDNRGATPLHYVAAASGHLEIVEVLLKAGADVNA

SEQ ID NO: 43 - protein, 33 aa, Artificial Sequence
  Ankyrin repeat module
KDNRGATPLHYAAASGHLEIVEVLLKAGADVNA

SEQ ID NO: 44 - protein, 34 aa, Artificial Sequence
  Ankyrin repeat module
KDTEYGWTPLHIAASDGHLEIVEVLLKAGADVNA

SEQ ID NO: 45 - protein, 33 aa, Artificial Sequence
  Ankyrin repeat module
KDYAGSTPLHAAAFVGHLEIVEVLLKAGADVNA

SEQ ID NO: 46 - protein, 33 aa, Artificial Sequence
  Ankyrin repeat module
KDLIGWTPLHLAAHYGHLEIVEVLLKAGADVNA

SEQ ID NO: 47 - protein, 33 aa, Artificial Sequence
  Ankyrin repeat module
KDAQGWTPLHIAAWEGHLEIVEVLLKAGADVNA

SEQ ID NO: 48 - protein, 33 aa, Artificial Sequence
  Ankyrin repeat module
KDWTGETPLHLAAFEGHLEIVEVLLKAGADVNA

SEQ ID NO: 49 - protein, 33 aa, Artificial Sequence
  Ankyrin repeat module
KDHQGETPLHLAASQGHLEIVEVLLKAGADVNA

SEQ ID NO: 50 - protein, 33 aa, Artificial Sequence
  Ankyrin repeat module
KDLAGYTPLHLAAWTGHLEIVEVLLKAGADVNA

SEQ ID NO: 51 - protein, 33 aa, Artificial Sequence
  Ankyrin repeat module
KDQWGRTPLHIAAASGHLEIVEVLLKAGADVNA

SEQ ID NO: 52 - protein, 124 aa, Artificial Sequence
  Ankyrin repeat domain specific for human serum albumin
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 53 - protein, 124 aa, Artificial Sequence
  Ankyrin repeat domain specific for human serum albumin
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 54 - protein, 124 aa, Artificial Sequence
  Ankyrin repeat domain specific for human serum albumin
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 55 - protein, 124 aa, Artificial Sequence
  Ankyrin repeat domain specific for CD3
DLGQKLLEAAWAGQDDEVRELLKAGADVNAKDSQGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 56 - protein, 124 aa, Artificial Sequence
  Ankyrin repeat domain specific for CD3
DLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 57 - protein, 124 aa, Artificial Sequence
  Ankyrin repeat domain specific for CD3
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 58 - protein, 124 aa, Artificial Sequence
  Ankyrin repeat domain specific for CD3
DLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 59 - protein, 124 aa, Artificial Sequence
  Ankyrin repeat domain specific for CD3
DLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKLN

SEQ ID NO: 60 - protein, 242 aa, Artificial Sequence
  CD33 target protein (ECD)
DPNFWLQVQESVTVQEGLCVLVPCTFFHPIPYYDKNSPVHGYWFREGAIISGDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 61 - protein, 120 aa, Artificial Sequence
  CD33 target protein (ECD splice variant)
DLTHRPKILIPGTLEPGHSKNLTCSVSWASEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 62 - protein, 33 aa, Artificial Sequence
  Ankyrin repeat module
  VARIANT 1,3,4,6,13,14,15,17,18,19,22,26,27: wherein Xaa denotes any amino acid (preferably not cysteine, glycine, or proline)
XDXXGXTPLHLAXXXGXXXLVXVLLXXGADVNA

SEQ ID NO: 63 - protein, 33 aa, Artificial Sequence
  Ankyrin repeat module
  VARIANT 1,3,4,6,14,15,27: wherein Xaa in positions 1, 3, 4, 6, 14, 15 denotes any amino acid (preferably not cysteine, glycine, or proline), and Xaa in position 27 is selected from the group consisting of asparagine, histidine, or tyrosine
XDXXGXTPLHLAAXXGHLEIVEVLLKXGADVNA

SEQ ID NO: 64 - protein, 127 aa, Artificial Sequence
  Ankyrin repeat domain specific for CD33
GSDLGWKLLLAASRGQDDEVRILLAAGADVNAKDIDEGYTPLHIAAYYGHLEIVEVLLKAGADVNAKDRYGKTPLHLAAISGHEDIAEVLLKAGADVNAQDDKGDTPADLAADYGHEDIAEVLQKAA

SEQ ID NO: 65 - protein, 24 aa, Artificial Sequence
  PT-rich peptide linker
GSPTPTPTTPTPTPTTPTPTPTGS

SEQ ID NO: 66 - protein, 22 aa, Artificial Sequence
  PT-rich peptide linker
GSPTPTPTTPTPTPTTPTPTPT

SEQ ID NO: 67 - protein, 5 aa, Artificial Sequence
  Consensus GS linker
  VARIANT 1..5: [Gly-Gly-Gly-Gly-Ser]n, wherein n is 1, 2, 3, 4, 5, or 6
GGGGS

SEQ ID NO: 68 - protein, 10 aa, Artificial Sequence
  His-tag
MRGSHHHHHH

SEQ ID NO: 69 - protein, 30 aa, Artificial Sequence
  N-cap
DLGKKLLQAARAGQLDEVRELLKAGADVNA

SEQ ID NO: 70 - protein, 30 aa, Artificial Sequence
  N-cap
DLGKKLLQAARAGQLDEVRILLKAGADVNA

SEQ ID NO: 71 - protein, 30 aa, Artificial Sequence
  N-cap
DLGKKLLQAARAGQLDEVRILLAAGADVNA

SEQ ID NO: 72 - protein, 30 aa, Artificial Sequence
  N-Cap (randomized)
  VARIANT 4,5,11,12,17,20,22,23,24: wherein Xaa denotes any amino acid (preferably not cysteine, glycine, or proline)
DLGXXLLQAAXXGQLDXVRXLXXXGADVNA

SEQ ID NO: 73 - protein, 28 aa, Artificial Sequence
  C-cap
QDKFGKTPADIAADNGHEDIAEVLQKLN

SEQ ID NO: 74 - protein, 28 aa, Artificial Sequence
  C-cap
QDKSGKTPADLAARAGHQDIAEVLQKAA

SEQ ID NO: 75 - protein, 28 aa, Artificial Sequence
  C-cap
QDSSGFTPADLAALVGHEDIAEVLQKLA

SEQ ID NO: 76 - protein, 28 aa, Artificial Sequence
  C-cap (randomized)
  VARIANT 1,3,4,6,9,10,11,15,17,19,20,21,22,23,24,25,26: wherein Xaa denotes any amino acid (preferably not cysteine, glycine, or proline)
XDXXGXTPXXXAARXGXQXXXXXXXXAA

SEQ ID NO: 77 - protein, 125 aa, Artificial Sequence
  DARPin protein #29
DLGVKLLLAAERGQLDEVRILLKAGADVNAKDIAEGYTPLHIAAYQGHLEIVEVLLKAGADVNAKDRYGKTPLHLAAIGGHLEIVEVLLKAGADVNAQDNKGSTPADLAADYGHQDIAEVLQKAA

SEQ ID NO: 78 - protein, 125 aa, Artificial Sequence
  DARPin protein #30
DLGKKLLLAAERGQLDEVRILLKAGADVNAKDRAEGYTPLHIAAYQGHLEIVEVLLKAGADVNAKDRYGKTPLHLAAISGHLEIVEVLLKAGADVNAQDNKGSTPADLAADYGHQDIAEVLQKAA

SEQ ID NO: 79 - protein, 34 aa, Artificial Sequence
  Ankyrin repeat module
KDIAEGYTPLHIAAYQGHLEIVEVLLKAGADVNA

SEQ ID NO: 80 - protein, 33 aa, Artificial Sequence
  Ankyrin repeat module
KDRYGKTPLHLAAIGGHLEIVEVLLKAGADVNA

SEQ ID NO: 81 - protein, 34 aa, Artificial Sequence
  Ankyrin repeat module
KDRAEGYTPLHIAAYQGHLEIVEVLLKAGADVNA

SEQ ID NO: 82 - protein, 273 aa, Artificial Sequence
  DARPin protein #31
DLGVKLLLAAERGQLDEVRILLKAGADVNAKDIAEGYTPLHIAAYQGHLEIVEVLLKAGADVNAKDRYGKTPLHLAAIGGHLEIVEVLLKAGADVNAQDNKGSTPADLAADYGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 83 - protein, 273 aa, Artificial Sequence
  DARPin protein #32
DLGKKLLLAAERGQLDEVRILLKAGADVNAKDRAEGYTPLHIAAYQGHLEIVEVLLKAGADVNAKDRYGKTPLHLAAISGHLEIVEVLLKAGADVNAQDNKGSTPADLAADYGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 84 - protein, 273 aa, Artificial Sequence
  DARPin protein #33
DLGVKLLLAASRGQLDEVRILLKAGADVNAKDIDEGYTPLHIAAYYGHLEIVEVLLKAGADVNAKDRYGKTPLHLAAISGHLEIVEVLLKAGADVNAQDDKGDTPADLAADYGHQDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 85 - protein, 12 aa, Artificial Sequence
  His- tag
MRGSHHHHHHGS